(19) United States
(12) Patent Application Publication
(10) Pub. No.: US 2022/0151292 A1
(43) Pub. Date: May 19, 2022

(54) NICOTINE POUCH COMPOSITION

(71) Applicant: NCP NextGen A/S, Vejle (DK)

(72) Inventors: Kent Albin Nielsen, Brande (DK); Jessie Poulsen, Odense SV (DK); My Ly Lao Stahl, Vejle Ost (DK); Bine Hare Jakobsen, Ry (DK)

(21) Appl. No.: 17/099,446

(22) Filed: Nov. 16, 2020

(51) Int. Cl. A24F 23/02; A24B 13/00; A61K 31/465

(52) U.S. Cl. CPC A24F 23/02; A61K 31/465; A24B 13/00

(57) ABSTRACT

A pouch composition is disclosed, the pouch composition includes a nicotine-ion exchange resin combination, water in an amount of at least 15% by weight of the pouch composition, and inorganic divalent cations. Also, an oral pouched nicotine product can include a saliva-permeable pouch and the pouch composition in said pouch.

### FIELD OF THE INVENTION

The present invention relates to pouch compositions and an oral pouched nicotine product according to the claims.

### BACKGROUND OF THE INVENTION

Delivery of nicotine by smoking has many well-known drawbacks, in particular health related problems, such as inclusion of carcinogenic substances.

However, tobacco substitutes also suffer from disadvantages, such as inadequate relief of cravings for the user.

A further challenge in the prior art is that the desired release of nicotine should be attractive to the user of the pouch from a user perspective.

Yet at further challenge in relation to the prior art may be that pouches as delivery vehicle for nicotine may be somewhat costly and thereby impose restrictions on the way pouches are designed in order to keep manufacturing costs in check.

It is an object of one embodiment of the present invention to provide a nicotine containing pouch, e.g. as a tobacco substitute, which may solve the above problems.

### SUMMARY OF THE INVENTION

The present invention relates to a pouch composition comprising
a nicotine-ion exchange resin combination,
water in an amount of at least 15% by weight of the pouch composition, and
inorganic divalent cations.

One advantage of the present invention may be that a relatively high stability of the provided nicotine may be obtained, while at the same time obtaining a relatively fast nicotine release. Obtaining a high stability may lead to nicotine being bound too effectively e.g. to a carrier and therefore lead to slow release. By means of the claimed pouch composition, including combination of a water content of at least 15% by weight of the composition and divalent inorganic cations, a high stability yet fast release is facilitated, while also having a very desirable mouthfeel and taste. The high water content facilitates effective release of nicotine during use.

One advantage of the invention is that a relatively fast release rate of nicotine from the pouch composition may be obtained due to the presence of the divalent cations. At the same time a desirable moist mouthfeel is provided, due to the high water content, which also facilitate fast nicotine release.

Furthermore, the invention may advantageously provide a more effective release of nicotine during use of a pouch comprising the pouch composition. Obtaining an effective release of nicotine may enable a lower total dose of nicotine with the same amount of nicotine released, due to a minimization of any residual nicotine not released from the pouch composition.

In an advantageous embodiment of the invention, the solid oral nicotine formulation comprises inorganic divalent cations in molar ratio of at least 0.1 relative to the amount of nicotine in the nicotine-ion exchange resin combination, such as at least 0.25 relative to the amount of nicotine in the nicotine-ion exchange resin combination, such as at least 0.5 relative to the amount of nicotine in the nicotine-ion exchange resin combination.

The amount of divalent cations should advantageously be high enough to enable ion-exchange of the complexed nicotine for the divalent cations during use of a pouch comprising the pouch composition.

Furthermore, the amount of inorganic divalent cations may advantageously also decrease the probability of exchanged nicotine from re-complexing with the ion-exchange resin, simply by occupying binding sites on the ion-exchange resin during use.

In an embodiment of the invention the amount of inorganic divalent cations may even prevent exchanged nicotine from re-complexing with the ion-exchange resin during use.

Also, the amount of inorganic divalent cations may decrease the probability of any un-complexed nicotine, such as free base nicotine and/or exchanged nicotine from complexing/re-complexing with the ion-exchange resin during use.

In an advantageous embodiment of the invention, the solid oral nicotine formulation comprises inorganic divalent cations in a molar ratio of at most 5 relative to the amount of nicotine in the nicotine-ion exchange resin combination, such as at most 3.75 relative to the amount of nicotine in the nicotine-ion exchange resin combination, such as at most 2.5 relative to the amount of nicotine in the nicotine-ion exchange resin combination.

One advantage of the above embodiment may be that including inorganic divalent cations in a not too high amount facilitates a desirable taste and mouthfeel, by avoiding or minimizing undesirable taste and/or mouthfeel, such as undesired salty taste, a local dehydration or even an oral dehydrating sensation.

In an embodiment of the invention the pouch composition comprises inorganic divalent cations in a molar ratio of between 0.1 and 5.0 relative to the amount of nicotine in the nicotine-ion exchange resin combination, such as between 0.1 and 4.0 relative to the amount of nicotine in the nicotine-ion exchange resin combination, such as between 0.1 and 3.0 relative to the amount of nicotine in the nicotine-ion exchange resin combination, such as between 0.1 and 2.0 relative to the amount of nicotine in the nicotine-ion exchange resin combination, such as between 0.1 and 1.0 relative to the amount of nicotine in the nicotine-ion exchange resin combination.

In an embodiment of the invention the pouch composition comprises inorganic divalent cations in a molar ratio of between 0.1 and 5.0 relative to the amount of nicotine in the nicotine-ion exchange resin combination, such as between 0.5 and 5.0 relative to the amount of nicotine in the nicotine-ion exchange resin combination, such as between 0.75 and 5.0 relative to the amount of nicotine in the nicotine-ion exchange resin combination, such as between 1.0 and 4.0 relative to the amount of nicotine in the nicotine-ion exchange resin combination, such as between 2.0 and 4.0 relative to the amount of nicotine in the nicotine-ion exchange resin combination.

In an embodiment of the invention the pouch composition comprises inorganic divalent cations in a molar ratio of between 0.01 and 5.0 relative to the amount of nicotine in the nicotine-ion exchange resin combination, such as between 0.01 and 4.0 relative to the amount of nicotine in the nicotine-ion exchange resin combination, such as between 0.01 and 3.0 relative to the amount of nicotine in the nicotine-ion exchange resin combination, such as between 0.01 and 2.0 relative to the amount of nicotine in the nicotine-ion exchange resin combination, such as between 0.01 and 1.0 relative to the amount of nicotine in the nicotine-ion exchange resin combination.

Here, the molar ratio refers to the molar content of divalent cations divided by the molar content of nicotine.

In an advantageous embodiment of the invention, the inorganic divalent cations are selected from the group consisting of divalent cations of calcium, magnesium, iron, zinc, and any combination thereof.

In an advantageous embodiment of the invention, the inorganic divalent cations are selected from the group consisting of divalent cations of calcium and magnesium.

In an embodiment of the invention, the inorganic divalent cations are provided as a salt comprising inorganic or organic anions.

In an advantageous embodiment of the invention, the inorganic divalent cations are provided as a salt comprising anions selected from the group consisting of carboxylates, such as acetate, lactate, oxalate, propionate, or levulinate; organic sulfonate; organic sulfate; organic phosphate; chloride, bromide, nitrate, sulfate, hydrogen phosphate, oxide, and any combination thereof.

In an embodiment of the invention the inorganic divalent cations are provided as a salt in an amount of between 0.1 and 15.0% by weight of the composition, such as between 0.1 and 10.0% by weight of the composition, such as between 0.5 and 10.0% by weight of the composition.

In an embodiment of the invention, the organic anions are selected from the group consisting of carboxylates, such as acetate, lactate, oxalate, propionate, levulinate; organic sulfonate; organic sulfate; organic phosphate; and any combination thereof.

In an advantageous embodiment of the invention, the inorganic divalent cations are provided as an inorganic salt.

In an advantageous embodiment of the invention, the inorganic divalent cations are provided as an inorganic salt in an amount of between 0.1 and 15.0% by weight of the composition, such as between 0.1 and 10.0% by weight of the composition, such as between 0.5 and 10.0% by weight of the composition.

In an embodiment of the invention the inorganic divalent cations are provided as an inorganic salt in the amount of between 0.1 and 15.0% by weight of the composition, such as between 0.1 and 10.0% by weight of the composition, such as between 0.5 and 10.0% by weight of the composition.

In an embodiment of the invention the inorganic divalent cations are provided as an inorganic salt in the amount of between 0.1 and 15.0% by weight of the composition, such as between 0.1 and 10.0% by weight of the composition, such as between 0.5 and 7.0% by weight of the composition, such as between 0.1 and 7.0% by weight of the composition, such as between 0.5 and 5.0% by weight of the composition, such as between 0.5 and 4.0% by weight of the composition.

In an advantageous embodiment of the invention, inorganic divalent cations are provided as an inorganic salt comprising inorganic anions selected from the group consisting of chloride, bromide, nitrate, sulfate, hydrogen carbonate, hydrogen phosphate, oxide, hydroxide, and any combination thereof.

It is noted that in some embodiments, the inorganic anions may be combined e.g. such that the cations form separate salts with two different types of anions. One example could e.g. be magnesium chloride combined with magnesium bromide.

In an advantageous embodiment of the invention, wherein the inorganic divalent cations are provided as an inorganic salt comprising inorganic anions are selected from the group consisting of chloride, bromide, sulfate, hydrogen carbonate, and any combination thereof.

In an advantageous embodiment of the invention, wherein the inorganic divalent cations are provided as an inorganic salt comprising inorganic anions are selected from the group consisting of chloride, bromide, sulfate, and any combination thereof.

In an advantageous embodiment of the invention, wherein the inorganic divalent cations are provided as an inorganic salt comprising inorganic anions are selected from the group consisting of chloride, bromide, and any combination thereof.

In an advantageous embodiment of the invention, the inorganic anions comprise chloride.

In an embodiment of the invention, the inorganic cations are magnesium and/or calcium and the anions comprise chloride.

In an embodiment of the invention, the inorganic anions are chloride.

In an embodiment of the invention, the inorganic cations are magnesium and/or calcium and the anions are chloride.

In an advantageous embodiment of the invention, the inorganic divalent cations are provided as an inorganic salt selected from the group consisting of calcium chloride or magnesium chloride, or combinations thereof.

In an embodiment of the invention, the divalent cations are provided as a pharmaceutically acceptable salt.

In an embodiment of the invention, the divalent cations are provided as a pharmaceutically acceptable inorganic salt.

In an embodiment of the invention the inorganic divalent cations are provided as a hydrated salt.

In an embodiment of the invention the inorganic divalent cations are provided as a hydrated inorganic salt.

In an embodiment of the invention, the divalent cations are provided as an alimentary acceptable salt.

In an embodiment of the invention, the divalent cations are provided as an alimentary acceptable inorganic salt.

In an advantageous embodiment of the invention, the divalent cations are provided as a water-soluble salt having a water-solubility of at least 5 gram per 100 mL of water measured at 25 degrees Celsius, atmospheric pressure and pH 7.0.

With atmospheric pressure is understood a pressure around 101.3 kPa or a pressure within the range of 90 to 110 kPa.

In an embodiment of the invention the inorganic divalent cations are provided as a water-soluble salt in the amount of between 0.1 and 15.0% by weight of the composition.

In an embodiment of the invention, the divalent cations are provided an inorganic and water-soluble salt having a water-solubility of at least 5 gram per 100 mL of water measured at 25 degrees Celsius, atmospheric pressure and pH 7.0.

In an embodiment of the invention the inorganic divalent cations are provided as a water-soluble salt in the amount of between 0.1 and 15.0% by weight of the composition, such as between 0.1 and 10.0% by weight of the composition, such as between 0.5 and 7.0% by weight of the composition, such as between 0.1 and 7.0% by weight of the composition, such as between 0.5 and 5.0% by weight of the composition, such as between 0.5 and 4.0% by weight of the composition.

In an embodiment of the invention the inorganic divalent cations are provided as an inorganic and water-soluble salt in the amount of between 0.1 and 15.0% by weight of the composition, such as between 0.1 and 10.0% by weight of the composition, such as between 0.5 and 7.0% by weight of the composition, such as between 0.1 and 7.0% by weight of the composition, such as between 0.5 and 5.0% by weight of the composition, such as between 0.5 and 4.0% by weight of the composition.

With provided is here understood, that the inorganic cations are added to the composition as a salt.

By providing the divalent cations as a water-soluble salt, the dissolution of the salt into cations could advantageously be faster and more effective, whereby relative fast release of nicotine could be achieved.

In an advantageous embodiment of the invention, the pouch composition comprises nicotine in an amount of at least 0.1% by weight, such as least 0.2% by weight of the pouch composition.

In an embodiment of the invention, the pouch composition comprises nicotine in an amount of 0.1 to 5.0% by weight of the pouch composition, such as 0.2 to 4.0% by weight of the pouch composition, such as 1.0 to 2.0% by weight of the pouch composition.

In an advantageous embodiment of the invention, the pouch composition comprises nicotine-ion exchange combination in an amount of 0.1 to 20% by weight of the pouch composition.

In an embodiment of the invention, the pouch composition comprises nicotine-ion exchange combination in an amount of 0.1 to 20% by weight of the pouch composition, such as 1.0 to 15% by weight of the pouch composition, such as 3.0 to 15% by weight of the pouch composition, such as 5.0 to 15% by weight of the pouch composition.

In an embodiment of the invention, the pouch composition comprises nicotine-ion exchange combination in an amount of 0.1 to 20% by weight of the pouch composition, such as 1.0 to 15% by weight of the pouch composition, such as 1.0 to 10% by weight of the pouch composition, such as 3.0 to 10% by weight of the pouch composition.

In an advantageous embodiment of the invention, the nicotine-ion exchange resin combination comprises nicotine in an amount of between 5 and 50% by weight.

In an embodiment of the invention the nicotine-ion exchange resin combination comprises nicotine complexed with ion exchange resin, wherein the nicotine constitutes an amount of between 5 and 50% by weight of nicotine-ion exchange resin combination.

In an embodiment of the invention the nicotine-ion exchange resin combination consists of nicotine complexed with ion exchange resin, wherein the nicotine constitutes an amount of between 10 and 50% by weight of nicotine-ion exchange resin combination, such as between 10 and 40% by weight of nicotine-ion exchange resin combination, such as. between 10 and 30% by weight of nicotine-ion exchange resin combination, such as between 10 and 25% by weight of nicotine-ion exchange resin combination.

In an embodiment of the invention the nicotine-ion exchange resin combination comprises free-base nicotine mixed with ion exchange resin, wherein the nicotine constitutes an amount of between 5 and 50% by weight of nicotine-ion exchange resin combination.

In an embodiment of the invention the nicotine-ion exchange resin combination comprises free-base nicotine mixed with ion exchange resin, wherein the nicotine constitutes an amount of between 5 and 50% by weight of nicotine-ion exchange resin combination, such as between 10 and 50% by weight of nicotine-ion exchange resin combination, such as between 20 and 50% by weight of nicotine-ion exchange resin combination, such as between 25 and 50% by weight of nicotine-ion exchange resin combination, such as between 25 and 45% by weight of nicotine-ion exchange resin combination.

In an embodiment of the invention the nicotine-ion exchange resin combination comprises free-base nicotine mixed with ion exchange resin, wherein the nicotine constitutes an amount of between 5 and 40% by weight of nicotine-ion exchange resin combination, such as between 10 and 40% by weight of nicotine-ion exchange resin combination, such as between 10 and 35% by weight of nicotine-ion exchange resin combination, such as between 10 and 25% by weight of nicotine-ion exchange resin combination, such as between 10 and 15% by weight of nicotine-ion exchange resin combination.

In an advantageous embodiment of the invention, the nicotine-ion exchange resin combination comprises nicotine in an amount of between 5 and 50% by weight and ion-exchange resin in an amount between 10 and 95% by weight.

In an embodiment of the invention the nicotine-ion exchange resin combination comprises nicotine in an amount of between 5 and 50% by weight and ion-exchange resin in an amount between 10 and 95% by weight.

In an embodiment of the invention the nicotine-ion exchange resin combination comprises nicotine in an amount of between 10 and 30% by weight and ion-exchange resin in an amount between 20 and 90% by weight.

In an embodiment of the invention the nicotine-ion exchange resin combination consists of nicotine in an amount of between 10 and 30% by weight and ion-exchange resin in an amount between 70 and 90% by weight.

In an embodiment of the invention the nicotine-ion exchange resin combination is substantially free of water.

In an embodiment of the invention the nicotine-ion exchange resin combination further comprising a C3 sugar alcohol.

In an embodiment, the C3 sugar alcohol may be selected from glycerol, propylene glycol, and any combination thereof.

In an embodiment of the invention the nicotine-ion exchange resin combination further comprises glycerol.

In an embodiment of the invention, the nicotine-ion exchange resin combination further comprises glycerol in an amount of 0.1 to 50% by weight, such as 5 to 40% by weight, such as 5 to 30% by weight.

In an embodiment of the invention the nicotine-ion exchange resin combination comprises nicotine in an amount of between 5 and 50% by weight and ion-exchange resin in an amount between 20 and 75% by weight.

In an embodiment of the invention the nicotine-ion exchange resin combination comprises water in an amount of no more than 75% by weight, such as no more than 50% by weight, such as no more than 40% by weight, such as no more than 30% by weight, such as no more than 20% by weight, such as no more than 10% by weight, such as no more than 5% by weight.

In an advantageous embodiment of the invention, the ion exchange resin comprises one or more resin(s) selected from the group consisting of:
(i) a methacrylic, weakly acidic type of resin containing carboxylic functional groups,
(ii) a copolymer of methacrylic acid and divinylbenzene, said copolymer containing carboxylic functional groups,
(iii) a polystyrene, strongly acidic type of resin containing sulphonic functional groups,
(iv) a polystyrene, intermediate acidic type of resin containing phosphonic functional groups, and
(v) a combination thereof.

In an advantageous embodiment of the invention, the ion exchange resin comprises polacrilex resin.

In an advantageous embodiment of the invention, the ion exchange resin is polacrilex resin.

In an embodiment of the invention, the ion exchange resin is polacrilex resin.

In an embodiment of the invention, the polacrilex resin comprises or is Amberlite® IRP64.

In an advantageous embodiment of the invention, the nicotine-ion exchange resin combination comprises nicotine complexed with ion exchange resin.

In an advantageous embodiment of the invention, the nicotine-ion exchange resin combination is nicotine complexed with ion exchange resin.

Thus, in the above embodiment the nicotine-ion exchange resin combination consists of nicotine complexed with ion exchange resin.

In an advantageous embodiment of the invention, the nicotine-ion exchange resin combination comprises free-base nicotine mixed with ion exchange resin.

One advantage of the above embodiment may be providing sustained release of nicotine. At the same time, the release rate of nicotine is not too slow to give the user the craving relief desired.

In an embodiment of the invention, the nicotine-ion exchange resin combination is free-base nicotine mixed with ion exchange resin.

In an embodiment of the invention the pouch composition comprises further nicotine.

In an embodiment of the invention the pouch composition comprises further nicotine.

In an embodiment of the invention the pouch composition comprises further nicotine selected from the group consisting of a nicotine salt, nicotine free base, nicotine bound to an ion exchanger, such as an ion exchange resin, such as nicotine polacrilex resin, a nicotine inclusion complex or nicotine in any non-covalent binding; nicotine bound to zeolites; nicotine bound to cellulose, such as microcrystalline cellulose, or starch microspheres, and mixtures thereof.

In an advantageous embodiment of the invention, the pouch composition comprises water in an amount of 15-65% by weight of the composition, such as 15-60% by weight of the composition, such as 15-50% by weight of the composition, such as 20-50% by weight of the composition, such as 20-40% by weight of the composition.

In an embodiment of the invention, the pouch composition comprises water in an amount of 15-65% by weight of the composition, such as 20-65% by weight of the composition, such as 25-65% by weight of the composition.

In an embodiment of the invention, the pouch composition comprises water in an amount of 15-65% by weight of the composition, such as 15-60% by weight of the composition, such as 15-50% by weight of the composition, such as 15-40% by weight of the composition.

In an embodiment of the invention, the pouch composition comprises water in an amount of 15-60% by weight of the composition, such as 15-50% by weight of the composition, such as 15-40% by weight of the composition, such as 15-30% by weight of the composition.

In an embodiment of the invention, the pouch composition comprises water in an amount of 15-40% by weight of the composition.

The water may be added as a separate component to be fully or partly mixed into other components, such as fibers. E.g. when adding a nicotine ion-exchange combination consisting of a mixture of free base nicotine with ion exchange resin and water, a significant amount of water of the final pouch composition may come from the mixture. For example, if the final amount pouch composition comprises 5% water from the nicotine-ion exchange resin combination, then up to one third of the water in the pouch composition derives from the nicotine-ion exchange resin combination.

In an advantageous embodiment of the invention, the pouch composition comprises at least one sugar alcohol.

In an embodiment of the invention, xylitol, maltitol, mannitol, erythritol, isomalt, sorbitol, lactitol, and mixtures thereof is used as the at least one sugar alcohol. The at least one sugar alcohol may also comprise further sugar alcohols. As an example embodiment, hydrogenated starch hydrolysates may be used, which comprises a mixture of sorbitol, maltitol and further sugar alcohols.

Sugar alcohols may advantageously facilitate and induce salivation of the pouch composition, whereby dissolution of the inorganic divalent cations are achieved, and release of nicotine is obtained, such as release of nicotine from the ion-exchange resin and release of nicotine from the pouch.

Sugar alcohols may advantageously be used to further increase the nicotine release from the pouch.

Also, sugar alcohols may advantageously be used for obtaining a desirable mouthfeel by increasing salivation and thereby counteract any local dehydration or oral dehydrating sensation experienced by the user of the pouch.

Thus, sugar alcohol may advantageously be used in combination with inorganic divalent cations in order to achieve a desirable release of nicotine, while also a desirable taste is achieved.

In an embodiment of the invention, the at least one sugar alcohol is selected from sugar alcohols having at least 4 carbon atoms.

In an advantageous embodiment of the invention, the at least one sugar alcohol is selected from xylitol, maltitol, mannitol, erythritol, isomalt, sorbitol, lactitol, and mixtures thereof.

In an advantageous embodiment of the invention, the pouch composition comprises at least two sugar alcohols.

It is noted that different sugar alcohols may be applied for the purpose of taste and salivation, where the sugar alcohol composition is made of different sugar alcohols having different properties with respect to storage, bacteria growth, processability and/or taste.

In an embodiment of the invention, the at least two sugar alcohols are selected from xylitol, maltitol, mannitol, erythritol, isomalt, sorbitol, lactitol, and mixtures thereof.

In an advantageous embodiment of the invention, the pouch composition comprises sugar alcohol in an amount of at least 1% by weight of the composition, such as at least 2% by weight of the composition, such as at least 5% by weight of the composition, such as at least 10% by weight of the composition, such as at least 15% by weight of the composition.

In an advantageous embodiment of the invention, the pouch composition comprises sugar alcohol in an amount of 1 to 80% by weight of the composition, such as 2 to 70% by weight of the composition, such as 5 to 60% by weight of the composition, such as 10 to 50% by weight of the composition, such as 15 to 50% by weight of the composition.

In an embodiment of the pouch composition comprises sugar alcohol in an amount of 1 to 80% by weight of the composition, such as 2 to 70% by weight of the composition, such as 5 to 60% by weight of the composition, such as 10 to 50% by weight of the composition, such as 15 to 50% by weight of the composition.

In an embodiment of the pouch composition comprises sugar alcohol in an amount of 1 to 80% by weight of the composition, such as 10 to 70% by weight of the composition, such as 10 to 60% by weight of the composition, such as 15 to 60% by weight of the composition, such as 20 to 60% by weight of the composition, such as 20 to 50% by weight of the composition.

In an advantageous embodiment of the invention, the pouch composition comprises at least one water-insoluble fiber.

In an advantageous embodiment of the invention, the pouch composition comprises said water-insoluble fiber in an amount between 5 and 50% by weight of the pouch composition, such as 10-45% by weight of the pouch composition, such as 15-40% by weight of the pouch composition.

In an embodiment of the invention, the pouch composition comprises said water-insoluble fiber in an amount between 5 and 50% by weight of the pouch composition, such as 5-45% by weight of the pouch composition, such as 5-40% by weight of the pouch composition.

In an embodiment of the invention, the pouch composition comprises said water-insoluble fiber in an amount between 5 and 50% by weight of the pouch composition, such as 10-50% by weight of the pouch composition, such as 15-50% by weight of the pouch composition.

An advantage of the above embodiment may be that a residue is left even after use of a nicotine pouch comprising the pouch composition. This may lead to a pleasant perception for users of the nicotine pouch, e.g. due to similarity with tobacco containing products.

The water-insoluble fiber may advantageously provide a desirable mouthfeel throughout the use of the pouch.

In an advantageous embodiment of the invention, the water-insoluble fiber is a plant fiber.

In an advantageous embodiment of the invention, the water-insoluble fiber is selected from wheat fibers, pea fibers, rice fiber, maize fibers, oat fibers, tomato fibers, barley fibers, rye fibers, sugar beet fibers, buckwheat fibers, potato fibers, cellulose fibers, apple fibers, cocoa fibers, cellulose fibers, bran fibers, bamboo fibers, powdered cellulose, and combinations thereof.

Powdered cellulose within the scope of the invention is understood to be cellulose prepared by processing alpha-cellulose obtained as a pulp from strains of fibrous plant materials, such as wood pulp.

In an embodiment of the invention, the water-insoluble fiber comprises or consists of cereal fibers.

In an embodiment of the invention, the water-insoluble fiber comprises or consists of fruit and/or vegetable fibers.

In an embodiment of the invention, the water-insoluble composition comprises or consists of water-insoluble fiber selected from wheat fibers, oat fibers, pea fibers, powdered cellulose, or combinations thereof.

In an embodiment of the invention, the water-insoluble composition comprises or consists of water-insoluble fiber selected from wheat fibers, oat fibers, pea fibers, or combinations thereof.

In an embodiment of the invention, the water-insoluble composition comprises or consists of water-insoluble fiber selected from wheat fibers, oat fibers, or combinations thereof.

Non-limiting examples of usable water-insoluble fibers include Vitacel WF 600, Vitacel HF 600, Vitacel P95, Vitacel WF 200, Vitacel L00, Vitacel Erbsenfaser EF 150, Vitacel bamboo fiberbaf 90, Vitacel HF 600, Vitacel Cellulose L700G, Vitacel PF200, Vitacel potatofiber KF200, Vitacel bamboo fiberhaf BAF40, Vitacel Haferfaser/oat fiber HF-401-30 US.

Non-limiting examples of usable powdered cellulose include Vitacel L 00, Vitacel Cellulose L700G, Vitacel LC1000, Vitacel L600-20, Vitacel L600 etc.

In an embodiment, the powdered cellulose is chemically unmodified. Thus, powdered cellulose may be chemically unmodified cellulose fibers, which do not include e.g. microcrystalline cellulose (MCC).

In an advantageous embodiment of the invention, the water-insoluble fiber has a water binding capacity of at least 200%, such as at least 300%, such as at least 400%.

An advantage of the above embodiment may be that the high water-binding capacity enables pouch compositions having a high water-content.

Furthermore, the pouches having a high water-content where found to have a desirable texture and mouthfeel may while still being able to store manufactured pouches together in abutment e.g. in cans etc. without sticking too much together to result in ruptures of the pouches when being removed.

Also, water-insoluble fibers having a high water-binding capacity may reduce any nicotine exchange induced by the divalent cations happening prior to the pouch being used.

Hence, pouches comprising water-insoluble fibers having a high water-binding capacity could advantageously have a decreased relative standard deviation (RSD) on the nicotine content.

In an advantageous embodiment of the invention, the content of nicotine between a series of at least 10 oral pouches comprising said pouch composition holds a relative standard deviation (RSD) below 10%, preferably below 8%, more preferably at most 6%, even more preferably at most 4%, most preferably at most 2%.

In an embodiment of the invention, the content of the nicotine between a series of at least 10 oral pouches comprising said pouch composition holds a relative standard deviation (RSD) of 0.1-10%, preferably 0.1-8%, more preferably 0.1-6%, even more preferably 0.1-4%, and most preferably 0.1-2%.

In an embodiment of the invention, the water-insoluble fiber has a water binding capacity of 300 to 1500%, such as 400 to 1300%.

In an embodiment of the invention, the water-insoluble fiber has a water binding capacity of 200% to 1500%, such as 300 to 1300%, such as 200 to 800%, such as 300 to 800%, such as 400 to 600%.

In an embodiment of the invention, the water-insoluble fiber has a water binding capacity of 200 to 1500%, such as 300 to 1300%, such as 300 to 900%, such as 300 to 700%, such as 400 to 700%.

In an embodiment of the invention, the water-insoluble fiber has a water binding capacity of 200 to 1500%, such as 400 to 1500%, such as 500 to 1500%, such as 500 to 1200%, such as 500 to 1000%.

In an embodiment of the invention, the water-insoluble fiber has a swelling capacity of at least 5.0 mL/g, such as 5.0-20 mL/g.

An advantage of the above embodiment is that the amount of water-insoluble fiber can be reduced without compromising the mouthfeel during use. If an amount of water-insoluble fiber is substituted for a water-soluble component, the swelling of the water-insoluble fiber will during use counteract the dissolution of the water-soluble component, thereby the user will not experience any decrease in pouch content during use.

In an embodiment of the invention, the water-insoluble fibers are selected from pea fibers, powdered cellulose, and combinations thereof, and wherein the pouch composition comprises flavor in an amount of no more than 10% by weight of the pouch composition.

In an embodiment of the invention, the pouch composition comprises water-insoluble fibers selected from pea fibers and powdered cellulose, or a combination thereof, and flavor in an amount of 0.01-10% by weight of the pouch composition.

In an advantageous embodiment of the invention, the water-insoluble fiber has a density of 50 to 500 gram per Liter, such as 100 to 400 gram per Liter, such as 200 to 300 gram per Liter.

The use of water-insoluble fiber having a relatively low bulk density, will provide not only a good mouthfeel, but also an effective release from the pouch, due to the fact that a relatively low bulk density promotes effective salivation, thereby dissolution and release of water-soluble ingredients of the composition.

In an advantageous embodiment of the invention, the pouch composition comprises a pH regulating agent.

In an advantageous embodiment of the invention, the pouch composition comprises pH regulating agent in an amount between 0.01 and 15% by weight of the pouch composition, such as between 0.5 and 10% by weight of the pouch composition, such as between 1 and 10% by weight of the pouch composition, such as between 5 and 10% by weight of the pouch composition.

Obtaining a relatively fast release rate of nicotine and an effective uptake/absorption may be desirable as this ensures a fast effect for the user, i.e. craving relief.

Furthermore, the combination of having an effective release and an effective absorption advantageously enables a relative high exploitation of the nicotine dose within the pouch. Having a relative high exploitation of the nicotine dose within the pouch may further provide a reduction of necessary nicotine dose of the pouch, without compromising the resulting effect. A lower nicotine dose may in tern result in a reduction in production cost, as nicotine may be relatively expensive, but may also assist users who want to lower their intake of nicotine.

In an advantageous embodiment of the invention, the pH regulating agent is a basic pH regulating agent, such as a basic buffering agent.

In an advantageous embodiment of the invention, the pH regulating agent is a buffering agent, such as a basic buffering agent.

In an embodiment of the invention, the pouch composition is adapted to give a pH of at least 8.0, such as a pH of at least 9.0, when 2.0 gram of pouch composition is added to 20 mL of 0.02 M potassium dihydrogen phosphate-buffer (pH adjusted to 7.4).

An advantage of the above embodiment may be that a relatively effective uptake of nicotine is facilitated due to the high pH value obtained.

A further advantage of the above embodiment may be that the need for preservative may be decreased or even eliminated and that low amounts of such preservatives may be used if not absent.

Also, the high pH value obtained may advantageously provide for a tingling sensation in the mouth which may be perceived as a desirable mouthfeel, e.g. due to resemblance with tobacco-based pouch products.

In an embodiment of the invention, the pH regulating agent is selected from the group consisting of Acetic acid, Adipic acid, Citric acid, Fumaric acid, Glucono-δ-lactone, Gluconic acid, Lactic acid, Malic acid, Maleic acid, Tartaric acid, Succinic acid, Propionic acid, Ascorbic acid, Phosphoric acid, Sodium orthophosphate, Potassium orthophosphate, Calcium orthophosphate, Sodium diphosphate, Potassium diphosphate, Calcium diphosphate, Pentasodium triphosphate, Pentapotassium triphosphate, Sodium polyphosphate, Potassium polyphosphate, Carbonic acid, Sodium carbonate, Sodium bicarbonate, Potassium carbonate, Calcium carbonate, Magnesium carbonate, Magnesium oxide, or any combination thereof.

In an advantageous embodiment of the invention, the pH regulating agent is selected from the group consisting Sodium carbonate, Sodium bicarbonate, Potassium carbonate, and Magnesium carbonate; Potassium bicarbonate; trometamol; phosphate buffer, or any combination thereof.

In an embodiment, the pouch composition comprises inorganic divalent cations, which may be provided as a water soluble salt, and in addition thereto a pH regulating agent selected from the group consisting Sodium carbonate, Sodium bicarbonate, Potassium carbonate, and Magnesium carbonate; Potassium bicarbonate; trometamol; phosphate buffer, or any combination thereof.

In the present context the term “trometamol” refers to (tris(hydroxymethyl)aminomethane), also sometimes referred to as tris buffer.

In an advantageous embodiment of the invention, the pH adjusting agent is selected from the group consisting of trometamol.

In an embodiment of the invention, the pH adjusting agent is trometamol.

In some embodiments, the pouch composition comprises humectant.

In an embodiment, the humectant is selected from the list of glycerol, propylene glycol, alginate, pectin, modified starch, hydroxypropyl cellulose, triacetin, polyethylene glycol (PEG), xanthan gum, and combinations thereof.

In an embodiment, the humectant is or comprises humectant in an amount of 0.5 to 10%, such as 0.5 to 5% by weight of the pouch composition, such as 1-3% by weight of the pouch composition.

In an embodiment, the humectant is or comprises alginate, such as sodium alginate, e.g. in an amount of 0.5 to 10%, such as 0.5 to 5% by weight of the pouch composition, such as 1-3% by weight of the pouch composition.

In an embodiment of the invention, the pouch composition is free of alginate.

In an embodiment of the invention the pouch composition is free of humectants consisting of alginate, pectin and xanthan gum.

In an advantageous embodiment of the invention, the pouch composition is adapted to release at least 30% nicotine within 10 minutes when exposed to in vitro conditions described in example 7A.

In an advantageous embodiment of the invention, the pouch composition is adapted to release at least 25% more nicotine within 5 minutes compared to a corresponding pouch composition without divalent cations when exposed to the in vitro conditions described in example 7A.

In an advantageous embodiment of the invention, the pouch composition comprises sodium chloride in an amount of 0.0-3.0% by weight of the pouch compositions, such as 0.05-1.0% by weight of the pouch composition, such as 0.1-1.0% by weight of the pouch composition.

Sodium chloride may advantageously be added in small amounts, i.e. 0.0-3.0% by weight as a flavor enhancer. Adding higher amounts of sodium chloride could induce an undesirable taste or mouthfeel.

In an advantageous embodiment of the invention, the pouch composition further comprises a preservative.

The preservative may help to preserve the pouch composition against undesirable microbiological growths.

In an advantageous embodiment of the invention, the pouch composition further comprises a preservative in an amount of 0.05 to 0.5% by weight of the pouch composition, such as 0.1 to 0.2% by weight of the pouch composition.

Non-limiting examples of usable preservatives within the scope of the invention includes sorbic acid (E200) and salts thereof (e.g. sodium sorbate (E201), potassium sorbate (E202), calcium sorbate (E203)), benzoic acid (E210) and salts thereof (e.g. sodium benzoate (E211), potassium benzoate (E212), calcium benzoate (E213)).

In an advantageous embodiment of the invention, the pouch composition comprises less than 0.1% by weight of preservatives, such as less than 0.05% by weight of preservatives.

Thus, the pouch composition may comprise preservatives in an amount of 0 to 0.1% by weight of preservatives, such as in an amount of 0 to 0.05% by weight of preservatives. This includes zero content of preservatives, i.e. that the pouch composition is free of preservatives. The low amount or even absence of preservative may be realized by obtaining a relatively alkaline environment, particularly by the use of free-base nicotine.

In an advantageous embodiment of the invention, the pouch composition is free of preservatives.

In an advantageous embodiment of the invention, the pouch composition is a non-tobacco pouch composition.

In an advantageous embodiment of the invention, the pouch composition comprises less than 2.0% by weight of tobacco, such as less than 1.0% by weight of tobacco, such as less than 0.5% by weight of tobacco, such as 0.0% by weight of tobacco.

In an advantageous embodiment of the invention, the pouch composition comprises a non-tobacco fiber.

In an advantageous embodiment of the invention, the pouch composition is a powdered composition.

The invention further relates to an oral pouched nicotine product comprising a saliva-permeable pouch and the pouch composition of according to the invention or any of its embodiments enclosed in said pouch.

In an advantageous embodiment of the invention, the pouched nicotine product comprises nicotine in an amount of 0.5 to 20 mg, such as 1.0 to 20 mg, such as 5.0 to 15 mg.

In an advantageous embodiment of the invention, the pouched nicotine product comprises nicotine-ion exchange combination in an amount of 1 to 100 mg per pouch.

In an embodiment of the invention, the pouched nicotine product comprises nicotine-ion exchange combination in an amount of 1 to 100 mg per pouch, such as 10 to 90 mg per pouch, such as 10 to 80 mg per pouch, such as 20 to 80 mg per pouch, such as 30 to 80 mg per pouch, such as 40 to 80 mg per pouch, such as 50 to 80 mg per pouch.

In an embodiment of the invention, the pouched nicotine product comprises nicotine-ion exchange combination in an amount of 1 to 100 mg per pouch, such as 10 to 80 mg per pouch, such as 10 to 60 mg per pouch, such as 20 to 60 mg per pouch, such as 20 to 50 mg per pouch.

In an embodiment of the invention, the divalent cations are provided as a salt having a water-solubility of 5-500 grams per 100 mL of water measured at 25 degrees Celsius, atmospheric pressure and pH 7.0, such as 5-350 grams per 100 mL of water measured at 25 degrees Celsius, atmospheric pressure and pH 7.0.

In an embodiment of the invention, the inorganic divalent cations are provided as a salt in an amount of between 0.1 and 15.0% by weight of the composition, such as between 0.1 and 10.0% by weight of the composition, such as between 0.5 and 10.0% by weight of the composition, and the inorganic divalent cations are provided as an inorganic salt comprising inorganic anions selected from the group consisting of chloride, bromide, hydrogen carbonate, sulfate, and any combination thereof.

In an embodiment of the invention, the inorganic divalent cations are provided as a salt in an amount of between 0.1 and 15.0% by weight of the composition, such as between 0.1 and 10.0% by weight of the composition, such as between 0.5 and 10.0% by weight of the composition, and the divalent cations are provided as a water-soluble salt having a water-solubility of at least 5 gram per 100 mL of water measured at 25 degrees Celsius, atmospheric pressure and pH 7.0.

In an embodiment of the invention, the inorganic divalent cations are provided as an inorganic salt in an amount of between 0.1 and 15.0% by weight of the composition, such as between 0.1 and 10.0% by weight of the composition, such as between 0.5 and 10.0% by weight of the composition, and the water-soluble salt has a water-solubility of at least 5 gram per 100 mL of water measured at 25 degrees Celsius, atmospheric pressure and pH 7.0.

In an embodiment of the invention, the inorganic divalent cations are provided as a salt in an amount of between 0.1 and 15.0% by weight of the composition, such as between 0.1 and 10.0% by weight of the composition, such as between 0.5 and 10.0% by weight of the composition and the pouch composition comprises nicotine in an amount of at least 0.1% by weight, such as least 0.2% by weight of the pouch composition.

In an embodiment of the invention, the inorganic divalent cations are provided as a salt in an amount of between 0.1 and 15.0% by weight of the composition, such as between 0.1 and 10.0% by weight of the composition, such as between 0.5 and 10.0% by weight of the composition, the pouch composition comprises nicotine in an amount of at least 0.1% by weight, such as least 0.2% by weight of the pouch composition, and the solid oral nicotine formulation comprises inorganic divalent cations in a molar ratio of at most 5 relative to the amount of nicotine in the nicotine-ion exchange resin combination, such as at most 3.75 relative to the amount of nicotine in the nicotine-ion exchange resin combination, such as at most 2.5 relative to the amount of nicotine in the nicotine-ion exchange resin combination.

In an embodiment of the invention, the inorganic divalent cations are provided as a salt in an amount of between 0.1 and 15.0% by weight of the composition, such as between 0.1 and 10.0% by weight of the composition, such as between 0.5 and 10.0% by weight of the composition, and the pouch composition comprises nicotine-ion exchange combination in an amount of 0.1 to 20% by weight of the pouch composition.

In an embodiment of the invention, the inorganic divalent cations are provided as a salt in an amount of between 0.1 and 15.0% by weight of the composition, such as between 0.1 and 10.0% by weight of the composition, such as between 0.5 and 10.0% by weight of the composition, and the pouch composition comprises nicotine-ion exchange combination in an amount of 0.1 to 20% by weight of the pouch composition, and the solid oral nicotine formulation comprises inorganic divalent cations in a molar ratio of at most 5 relative to the amount of nicotine in the nicotine-ion exchange resin combination, such as at most 3.75 relative to the amount of nicotine in the nicotine-ion exchange resin combination, such as at most 2.5 relative to the amount of nicotine in the nicotine-ion exchange resin combination.

In an embodiment of the invention, the nicotine-ion exchange resin combination comprises nicotine in an amount of between 5 and 50% by weight and ion-exchange resin in an amount between 10 and 95% by weight, and the ion exchange resin is polacrilex resin.

In an embodiment of the invention, the pouch composition comprises nicotine-ion exchange combination in an amount of 0.1 to 20% by weight of the pouch composition, and the nicotine-ion exchange resin combination comprises nicotine in an amount of between 5 and 50% by weight and ion-exchange resin in an amount between 10 and 95% by weight, and the ion exchange resin is polacrilex resin.

In an embodiment of the invention, the inorganic divalent cations are provided as a salt in an amount of between 0.1 and 15.0% by weight of the composition, such as between 0.1 and 10.0% by weight of the composition, such as between 0.5 and 10.0% by weight of the composition, and the pouch composition comprises nicotine-ion exchange combination in an amount of 0.1 to 20% by weight of the pouch composition, and the pouch composition comprises water in an amount of 15-65% by weight of the composition, such as 15-60% by weight of the composition, such as 15-50% by weight of the composition, such as 20-50% by weight of the composition, such as 20-40% by weight of the composition.

In an embodiment of the invention, the at least one sugar alcohol is selected from xylitol, maltitol, mannitol, erythritol, isomalt, sorbitol, lactitol, and mixtures thereof, and the pouch composition comprises sugar alcohol in an amount of 1 to 80% by weight of the composition, such as 2 to 70% by weight of the composition, such as 5 to 60% by weight of the composition, such as 10 to 50% by weight of the composition, such as 15 to 50% by weight of the composition.

In an embodiment of the invention, the inorganic divalent cations are provided as a salt in an amount of between 0.1 and 15.0% by weight of the composition, such as between 0.1 and 10.0% by weight of the composition, such as between 0.5 and 10.0% by weight of the composition, and the pouch composition comprises nicotine-ion exchange combination in an amount of 0.1 to 20% by weight of the pouch composition, and the pouch composition comprises water in an amount of 15-65% by weight of the composition, such as 15-60% by weight of the composition, such as 15-50% by weight of the composition, such as 20-50% by weight of the composition, such as 20-40% by weight of the composition, and the pouch composition comprises sugar alcohol in an amount of 1 to 80% by weight of the composition, such as 2 to 70% by weight of the composition, such as 5 to 60% by weight of the composition, such as 10 to 50% by weight of the composition, such as 15 to 50% by weight of the composition.

In an embodiment of the invention, the pouch composition comprises sugar alcohol in an amount of 1 to 80% by weight of the composition, such as 2 to 70% by weight of the composition, such as 5 to 60% by weight of the composition, such as 10 to 50% by weight of the composition, such as 15 to 50% by weight of the composition, and the pouch composition comprises said water-insoluble fiber in an amount between 5 and 50% by weight of the pouch composition, such as 10-45% by weight of the pouch composition, such as 15-40% by weight of the pouch composition.

In an embodiment of the invention, the pouch composition comprises sugar alcohol in an amount of 1 to 80% by weight of the composition, such as 2 to 70% by weight of the composition, such as 5 to 60% by weight of the composition, such as 10 to 50% by weight of the composition, such as 15 to 50% by weight of the composition, and the pouch composition comprises said water-insoluble fiber in an amount between 5 and 50% by weight of the pouch composition, such as 10-45% by weight of the pouch composition, such as 15-40% by weight of the pouch composition, and the pouch composition comprises water in an amount of 15-65% by weight of the composition, such as 15-60% by weight of the composition, such as 15-50% by weight of the composition, such as 20-50% by weight of the composition, such as 20-40% by weight of the composition.

In an embodiment of the invention, the pouch composition comprises said water-insoluble fiber in an amount between 5 and 50% by weight of the pouch composition, such as 10-45% by weight of the pouch composition, such as 15-40% by weight of the pouch composition, and the water-insoluble fiber is selected from wheat fibers, pea fibers, rice fiber, maize fibers, oat fibers, tomato fibers, barley fibers, rye fibers, sugar beet fibers, buckwheat fibers, potato fibers, cellulose fibers, apple fibers, cocoa fibers, cellulose fibers, bran fibers, bamboo fibers, powdered cellulose, and combinations thereof.

In an embodiment of the invention, the inorganic divalent cations are provided as a salt in an amount of between 0.1 and 15.0% by weight of the composition, such as between 0.1 and 10.0% by weight of the composition, such as between 0.5 and 10.0% by weight of the composition, and the pouch composition comprises nicotine-ion exchange combination in an amount of 0.1 to 20% by weight of the pouch composition, and the pouch composition comprises water in an amount of 15-65% by weight of the composition, such as 15-60% by weight of the composition, such as 15-50% by weight of the composition, such as 20-50% by weight of the composition, such as 20-40% by weight of the composition, and the pouch composition comprises sugar alcohol in an amount of 1 to 80% by weight of the composition, such as 2 to 70% by weight of the composition, such as 5 to 60% by weight of the composition, such as 10 to 50% by weight of the composition, such as 15 to 50% by weight of the composition, and the pouch composition comprises said water-insoluble fiber in an amount between 5 and 50% by weight of the pouch composition, such as 10-45% by weight of the pouch composition, such as 15-40% by weight of the pouch composition.

In an embodiment of the invention, the pouch composition comprises pH regulating agent in an amount between 0.01 and 15% by weight of the pouch composition, such as between 0.5 and 10% by weight of the pouch composition, such as between 1 and 10% by weight of the pouch composition, such as between 5 and 10% by weight of the pouch composition, and the pH regulating agent is selected from the group consisting Sodium carbonate, Sodium bicarbonate, Potassium carbonate, and Magnesium carbonate; Potassium bicarbonate; trometamol; phosphate buffer, or any combination thereof.

In an embodiment of the invention, the pouch composition comprises pH regulating agent in an amount between 0.01 and 15% by weight of the pouch composition, such as between 0.5 and 10% by weight of the pouch composition, such as between 1 and 10% by weight of the pouch composition, such as between 5 and 10% by weight of the pouch composition, and the pH regulating agent is selected from the group consisting Sodium carbonate, Sodium bicarbonate, Potassium carbonate, and Magnesium carbonate; Potassium bicarbonate; trometamol; phosphate buffer, or any combination thereof, and the divalent cations are provided as a water-soluble salt having a water-solubility of at least 5 gram per 100 mL of water measured at 25 degrees Celsius, atmospheric pressure and pH 7.0.

In an embodiment of the invention, the pouch composition comprises pH regulating agent in an amount between 0.01 and 15% by weight of the pouch composition, such as between 0.5 and 10% by weight of the pouch composition, such as between 1 and 10% by weight of the pouch composition, such as between 5 and 10% by weight of the pouch composition, and the pH regulating agent is selected from the group consisting Sodium carbonate, Sodium bicarbonate, Potassium carbonate, and Magnesium carbonate; Potassium bicarbonate; trometamol; phosphate buffer, or any combination thereof, and the inorganic divalent cations are provided as an inorganic salt comprising inorganic anions selected from the group consisting of chloride, bromide, hydrogen carbonate, sulfate, and any combination thereof.

The invention further relates to a pouch composition comprising
a nicotine-ion exchange resin combination, and
inorganic multivalent cations.

In an advantageous embodiment of the invention, said multivalent cations are selected from the group consisting of multivalent ions of calcium, magnesium, zinc, aluminum, barium, iron, manganese, copper, lead, cobalt, nickel, such as Ca2+, Mg2+, Zn2+, Al3+, Ba2+, Fe2+, Fe3+, Fe4+, Mn2+, Mn4+, Cu4+, or any combinations thereof.

In an embodiment of the invention, the multivalent cations are selected from the group consisting of Ca2+, Mg2+, Zn2+, Ba2+, Fe2+, Fe3+, Fe4+, Al3+, Mn2+, Mn4+, Cu4+, and any combination thereof.

In an advantageous embodiment of the invention, the multivalent cations are selected from the group consisting of trivalent cations of aluminum, divalent cations of calcium, magnesium, iron, zinc, and any combination thereof.

In an advantageous embodiment of the invention, the multivalent cations are trivalent cations.

In an embodiment the trivalent cation is aluminum.

In an embodiment of the invention, the multivalent cations comprise aluminum chloride

In an embodiment of the invention, the multivalent cations are selected from the group consisting of aluminum chloride, divalent cations of calcium, magnesium, iron, zinc, and any combination thereof.

In an advantageous embodiment of the invention, the multivalent cations are selected from the group consisting of divalent cations of calcium, magnesium, iron, zinc, and any combination thereof.

In an advantageous embodiment of the invention, the multivalent cations are selected from the group consisting of divalent cations of calcium, magnesium, and any combination thereof.

### DETAILED DESCRIPTION OF THE INVENTION

As used herein the term “pouch composition” refers to the composition for use in an oral pouch, i.e. in pouches for oral use. Also, the terms “pouch composition” and “nicotine pouch composition” is used interchangeably.

As used herein the term “nicotine” refers to nicotine used as a refined/isolated substance. Particularly, nicotine does not refer to tobacco materials having a content of nicotine. Thus, when referring to nicotine amounts also to be understood as the nicotine dose, the amounts refers to the amount of pure nicotine.

Nicotine also covers nicotine not obtained from tobacco, often referred to as synthetic nicotine.

As used herein, a molar ratio refers to the ratio of the molar content of the first component divided by the molar content of the second component.

The relative content between the first component and the second component may also be presented as equivalents of the first component relative to the second component.

Thus, a pouch comprising divalent cations in a molar ratio of 0.1 relative to the amount of nicotine in the nicotine-ion exchange resin combination, may also be presented as a pouch comprising 0.1 eq. of divalent cations relative to the amount of nicotine in the nicotine-ion exchange resin combination, i.e. a pouch comprising 0.1 eq. of divalent cations and 1 eq. of nicotine in the nicotine-ion exchange resin combination.

As used herein the term “free-base nicotine” refers to non-protonated form of nicotine, and therefore does not include nicotine salts or nicotine provided as a complex between nicotine and an ion exchange resin. Nevertheless, the free-base nicotine may be mixed with an amount of ion exchange resin or water-soluble compositions such as sugar alcohols or water-soluble fibers. While free-base nicotine includes both free-base nicotine extracted from tobacco as well as synthetically manufactured free-base nicotine, the free-base nicotine is not provided in the form of tobacco or powdered tobacco. Typically, free-base nicotine is provided as a liquid.

As used herein the term “pouch” is intended to mean a container typically formed by a web of a fibrous material enclosing a cavity. The pouch is pouch designed for administration of an active ingredient in the oral cavity, and thus it is adapted for oral use, it is non-toxic and not water-soluble. The fibrous material may e.g. form a woven or non-woven web or fabric. The pouch may for example be sealed by bonding two corresponding pieces of web or fabric to each other along their edges to form a cavity for the nicotine and the non-water-soluble composition. In order to release the nicotine, the pouch is made water-permeable so as to allow saliva from the oral cavity to penetrate the pouch and enter the cavity, where the saliva can come into contact with the nicotine, whereby the nicotine are released from the oral pouch.

As used herein, the term “nicotine-ion exchange resin combination” refer to a combination comprising nicotine complexed with ion exchange resin and/or free-base nicotine mixed with ion exchange resin.

As used herein, the term “nicotine complexed with ion-exchange resin” refers to nicotine bound to an ion exchange resin.

In the present context, the term “free-base nicotine mixed with ion exchange resin” refers to a mixture comprising free-base nicotine and ion exchange resin. It is noted that even if some embodiments comprise a combination of nicotine complexed with ion exchange resin and nicotine in its free-base form mixed with ion exchange resin, the term “free-base nicotine mixed with ion exchange resin” requires the presence of nicotine in its free-base form. In some embodiments, the mixture is an aqueous mixture. Free-base nicotine and water is mixed with ion-exchange resin, whereby a mixture comprising both free-base nicotine and ion exchange resin is obtained. Free-base nicotine mixed with ion exchange resin is referred to as “premix” in the examples.

As used herein the term “powder composition” refers to composition in the form of powder, i.e. as a particulate material having a relatively small particle size, for example between 1 and 1200 micrometer. Particularly, by powder composition is not meant a powdered tobacco.

As used herein the term “humectant” is understood as a moistening agent used to keep pouches moist, i.e. a humectant is added to the pouch composition with the purpose of keeping the pouch moist. Hence, the term humectant does not refer to substances added for other purposes, hereunder also hygroscopic substances added for other purposes, such as sugar alcohols, water-insoluble fibers and glycerol associated with ion-exchange resin in nicotine-ion exchange resin combinations, such as nicotine polacrilex. Examples of humectants include alginate, propylene glycol, hydroxypropyl cellulose, and glycerol. It is noted that when glycerol is included as a humectant, the glycerol is added as free glycerol and therefore liquid at room temperature. Further examples of humectants include triacetin, modified starch, pectin, xanthan gum, etc. The term humectant does not refer to sugar alcohols comprising 4 or more carbons. Also, the term humectant does not refer to fibers, such as water-insoluble fiber, such as wheat fibers, pea fibers, rice fiber, maize fibers, oat fibers, tomato fibers, barley fibers, rye fibers, sugar beet fibers, buckwheat fibers, potato fibers, cellulose fibers, apple fibers, cocoa fibers, cellulose fibers, bran fibers, bamboo fibers, powdered cellulose, and combinations thereof. Also, the term humectant does not include e.g. NaCl.

As used herein the term “water-soluble” refers to a relatively high water-solubility, for example a water-solubility of more than 5 gram of water-soluble composition or substance per 100 mL of water measured at 25 degrees Celsius, atmospheric pressure and pH of 7.0. When referring to a “soluble” composition or substance, water-soluble is meant, unless otherwise stated.

As used herein the term “water-insoluble” refers to relatively low water-solubility, for example a water-solubility of less than 0.1 gram of composition or substance per 100 mL of water measured at 25 degrees Celsius, atmospheric pressure and pH of 7.0. When referring to “insoluble”, water-insoluble is meant unless otherwise stated. Therefore, compositions or substances having a water-solubility of between 0.1 and 5 gram per of composition or substance per 100 mL of water measured at 25 degrees Celsius, atmospheric pressure and pH of 7.0 are considered neither water-soluble nor water-insoluble, but having an intermediate water-solubility.

The pouches of the invention provide a nicotine release into the oral cavity. A release profile of nicotine may be obtained which both comprises a fast release period and a sustained release period.

As used herein the term “fast release” or “fast release period” may refer to the initial 2 minutes of the nicotine release profile, whereas the term “sustained release period refers” to the subsequent period of the release profile until end of experiment or end of use.

As used herein the term “fast release rate” refers to the released nicotine per minute within the initial 2 minutes.

As used herein the term “effective release” refers to the total release of nicotine over the release period of the experiment or the use period.

As used herein, the term “dissolve” is the process where a solid substance enters a solvent (such as oral saliva or water within the pouch) to yield a solution.

Typically, the pouches comprise openings, where the characteristic opening dimension is adapted to a characteristic dimension of the matrix composition so as to retain the matrix composition inside the pouch before use and/or to retain a part of the matrix composition, such as an water-insoluble composition, inside the pouch during use.

In order to obtain a pouch having suitable opening dimensions in view of the matrix composition to be used, the material for the pouch may be selected accordingly, e.g. comprising e.g. woven and/or non-woven fabric.

In other words, according to the various embodiments, the pouch forms a membrane allowing passage of saliva and prevents or inhibits passage of said matrix composition. The membrane of the pouch may be of any suitable material e.g. woven or non-woven fabric (e.g. cotton, fleece etc.), heat sealable non-woven cellulose or other polymeric materials such as a synthetic, semi-synthetic or natural polymeric material. An example of suitable pouch material is paper made of pulp and a small amount of wet strength agent. A material suitable for use must provide a semi-permeable membrane layer to prevent the powder or composition from leaving the bag or pouch during use. Suitable materials are also those that do not have a significant impact on the release of nicotine from the pouch.

The pouch composition is filled into pouches and is maintained in the pouch by a sealing. An ideal pouch is chemically and physically stable, it is pharmaceutically acceptable, it is insoluble in water, it is easy to fill with powder and seal, and it provides a semi-permeable membrane layer which prevent the powder from leaving the bag, but permit saliva and therein dissolved or sufficiently small suspended components from the pouch composition in the pouch, such as nicotine, to pass through said pouch.

The pouch may be placed in the oral cavity by the user. Saliva then enters into the pouch, and the nicotine and other components, which are soluble in saliva, start to dissolve and are transported with the saliva out of the pouch into the oral cavity, where the nicotine may be absorbed.

According to an embodiment of the invention, the pouch composition may further comprise one or more additives.

In an embodiment of the invention, said additives are selected from the group consisting of bile salts, cetomacrogols, chelating agents, citrates, cyclodextrins, detergents, enamine derivatives, fatty acids, labrasol, lecithins, phospholipids, synthetic and natural surfactants, nonionic surfactants, cell envelope disordering compounds, solvents, steroidal detergents, chelators, solubilization agents, charge modifying agents, pH regulating agents, degradative enzyme inhibitors, mucolytic or mucus clearing agents, membrane penetration-enhancing agents, modulatory agents of epithelial junction physiology, vasodilator agents, selective transport-enhancing agents, or any combination thereof. pH regulating agents include buffers.

In an embodiment of the invention, said additives are selected from the group consisting of cetylpyridinium chloride (CPC), benzalkonium chloride, sodium lauryl sulfate, polysorbate 80, Polysorbate 20, cetyltrimethylammonium bromide, laureth 9, sodium salicylate, sodium EDTA, EDTA, aprotinin, sodium taurocholate, saponins, bile salt derivatives, fatty acids, sucrose esters, azone emulsion, dextran sulphate, linoleic acid, labrafil, transcutol, urea, azone, nonionic surfactants, sulfoxides, sauric acid/PG, POE 23 lauryl ether, methoxysalicylate, dextran sulfate, methanol, ethanol, sodium cholate, Sodium taurocholate, Lysophosphatidyl choline, Alkylglycosides, polysorbates, Sorbitan esters, Poloxamer block copolymers, PEG-35 castor oil, PEG-hydrogenated castor oil, Caprocaproyl macrogol-8 glycerides, PEG-8 caprylic/capric, glycerides, Dioctyl sulfosuccinate, Polyethylene lauryl ether, Ethoxydiglycol, Propylene glycol, mono-di-caprylate, Glycerol monocaprylate, Glyceryl fatty acids (C.sub.8-C.sub.18) ethoxylated Oleic acid, Linoleic acid, Glyceryl caprylate/caprate, Glyceryl monooleate, Glyceryl monolaurate, Capryliccapric triglycerides, Ethoxylated nonylphenols, PEG-(8-50) stearates, Olive oil PEG-6, esters, Triolein PEG-6 esters, Lecithin, d-alpha tocopherol polyethylene glycol 1,000 succinate, Citric acid, Sodium citrate, BRIJ, Sodium laurate, 5-methoxysalicylic acid, Bile salts, Acetyl salicylate, ZOT, Docosahexaenoic acid, Alkylglycosides, Sodium glycocholate (GC-Na), Sodium taurocholate (TC-Na), EDTA, Choline salicylate, Sodium caprate (Cap-Na), N-lauryl-beta-D-maltopyranoside (LM), Diethyl maleate, Labrasol, Sodium salicylate, Mentol, Alkali metal alkyl sulphate, Sodium lauryl sulphate, Glycerin, Bile acid, Lecithin, phosphatidylcholine, phosphatidylserine, sphingomyelin, phosphatidylethanolamine, cephalin, lysolecithin, Hyaluronic acid: alkalimetal salts, sodium, alkaline earth and aluminum, Octylphenoxypolyethoxyethanol, Glycolic acid, Lactic acid, Chamomile extract, Cucumber extract, Borage oil, Evening primrose oil, Polyglycerin, Lysine, Polylysine, Triolein, Monoolein, Monooleates, Monolaurates, Polydocanol alkyl ethers, Chenodeoxycholate, Deoxycholate, Glycocholic acid, Taurocholic acid, Glycodeoxycholic acid, Taurodeoxycholic acid, Sodium glycocholate, Phosphatidylcholine, Phosphatidylserine, Sphingomyelin, Phosphatidylethanolamine, Cephalin, Lysolecithin, Alkali metal hyaluronates, Chitosan, Poly-L-arginine, Alkyl glucoside, Saccharide alkyl ester, Fusidic acid derivatives, Sodium taurdihydrofusidate (STDHF), L-α-phosphatidylcholine Didecanoyl (DDPC), Nitroglycerine, nitropruside, NOC5 [3-(2-hydroxy-l-(methyl-ethyl)-2-nitrosohydrazino)-l-propanamine], NOC12 [iV-ethyl-2-(l-ethyl-hydroxy-2-nitrosohydrazino)-ethanamine, SNAP [S-nitroso-N-acetyl-DL-penicillamine, NORI, NOR4, deacylmethyl sulfoxide, azone, salicylamide, glyceryl-l,3-diacetoacetate, l,2-isopropylideneglycerine-3-acetoacetate), Amino acids, Amino acid salts, monoaminocarboxlic acids, Glycine, alanine, phenylalanine, proline, hydroxyproline, hydroxyamino acids, serine, acidic amino acids, aspartic acid, Glutamic acid, Basic amino acids, Lysine, N-acetylamino acids, N-acetylalanine, N-acetylphenylalanine, TM-acetylserine, N-acetylglycine, N-acetyllysine, N-acetylglutamic acid, N-acetylproline, N-acetylhydroxyproline, lactic acid, malic acid and citric acid and alkali metal salts thereof, pyrrolidonecarboxylic acids, alkylpyrrolidonecarboxylic acid esters, N-alkylpyrrolidones, proline acyl esters, sodium lauryl phosphate, sodium lauryl sulphate, sodium oleyl phosphate, sodium myristyl sulphate, polyoxyethylene alkyl ethers, polyoxyethylene alkyl esters, and caproic acid, alkylsaccharide, fusidic acid, polyethylene glycol, cetyl alcohol, polyvinylpyrolidone, Polyvinyl alcohol, Lanolin alcohol, Sorbitan monooleate, Ethylene glycol tetraacetic acid, Bile acid conjugate with taurine, Cholanic acid and salts, Cyclodextran, Cyclodextrin, Cyclodextrin (beta), Hydroxypropyl-β-cyclodextran, Sulfobutylether-β-cyclodextran, Methyl-β-cyclodextrin, Chitosan glutamate, Chitosan acetate, Chitosan hydrochloride, Chitosan hydrolactate, 1-O-alkyl-2-hydroxy-sn-glycero-3-phosphocholine, 3-O-alkyl-2-acetoyl-sn-glycero-1-phosphocholine, 1-O-alkyl-2-O-acetyl-sn-glycero-3-phospho(N,N,N-trimethyl)hexanolamine, Propylene glycol, Tetradecylmaltoside (TDM), Sucrose dedecanoate.

As used herein, the term “pH regulating agent” refers to agents, which active adjust and regulates the pH value of the solution to which they have been added or are to be added. Thus, pH regulating agents may be acids and bases, including acidic buffering agents and alkaline buffering agents. On the other hand, pH regulating agents does not including substances and compositions that can only affect the pH by

dilution. Furthermore, pH regulating agents does not include e.g. flavoring, fillers, etc.

In an embodiment of the invention, said pH-regulating agents are selected from the group consisting of Acetic acid, Adipic acid, Citric acid, Fumaric acid, Glucono-δ-lactone, Gluconic acid, Lactic acid, Malic acid, Maleic acid, Tartaric acid, Succinic acid, Propionic acid, Ascorbic acid, Phosphoric acid, Sodium orthophosphate, Potassium orthophosphate, Calcium orthophosphate, Sodium diphosphate, Potassium diphosphate, Calcium diphosphate, Pentasodium triphosphate, Pentapotassium triphosphate, Sodium polyphosphate, Potassium polyphosphate, Carbonic acid, Sodium carbonate, Sodium bicarbonate, Potassium carbonate, Calcium carbonate, Magnesium carbonate, Magnesium oxide, or any combination thereof.

According to various embodiments of the invention, one or more sugar alcohols may be included in the pouch as part of the pouch composition, e.g. as a carrier or part thereof, or as a sweetener. Suitable sugar alcohols include sugar alcohols selected from the group of sorbitol, erythritol, xylitol, lactitol, maltitol, mannitol, hydrogenated starch hydrolyzates, isomalt, or any combination thereof.

In an embodiment of the invention the pouch composition comprises high intensity sweetener.

Preferred high intensity sweeteners include, but are not limited to sucralose, aspartame, salts of acesulfame, such as acesulfame potassium, alitame, saccharin and its salts, cyclamic acid and its salts, glycyrrhizin, dihydrochalcones, thaumatin, monellin, stevioside and the like, alone or in combination.

In an embodiment of the invention, the pouch composition comprises bulk sweeteners including sugar and/or sugarless components.

In an embodiment of the invention, the pouch composition comprises bulk sweetener in the amount of 1.0 to about 80% by weight of the pouch composition, more typically constitute 5 to about 70% by weight of the pouch composition, and more commonly 10 to 60% by weight of the pouch composition or 10-50% by weight of the pouch composition. Bulk sweeteners may function both as a sweetener and also as a humectant. In some embodiments, inclusion of certain ingredients may limit the about amounts of bulk sweetener further.

The sweeteners may often support the flavor profile of the pouch composition.

Sugar sweeteners generally include, but are not limited to saccharide-containing components commonly known in the art of pouches, such as sucrose, dextrose, maltose, saccharose, lactose, sorbose, dextrin, trehalose, D-tagatose, dried invert sugar, fructose, levulose, galactose, corn syrup solids, glucose syrup, hydrogenated glucose syrup, and the like, alone or in combination.

The sweetener can be used in combination with sugarless sweeteners. Generally, sugarless sweeteners include components with sweetening characteristics but which are devoid of the commonly known sugars and comprise, but are not limited to, sugar alcohols, such as sorbitol, mannitol, xylitol, hydrogenated starch hydrolyzates, maltitol, isomalt, erythritol, lactitol and the like, alone or in combination.

As used herein the term “flavor” is understood as having its ordinary meaning within the art. Flavor includes liquid and powdered flavors. Thus, flavors do of course not include sweeteners (such as sugar, sugar alcohols and high intensity sweeteners), or acids providing pure acidity/sourness, nor compounds providing pure saltiness (e.g. NaCl) or pure bitterness. Flavor enhancers include substances that only provide saltiness, bitterness or sourness. Flavor enhancers thus include e.g. NaCl, Citric acid, ammonium chloride etc.

The flavors can be natural or synthetic flavors.

In an embodiment of the invention the pouch composition comprises flavor. Flavor may typically be present in amounts between 0.01 and 15% by weight of the total composition of the pouch, such as between 0.01 and 5% by weight of the total composition.

Non-exhaustive examples of flavors suitable in embodiments of the present invention are coconut, coffee, chocolate, vanilla, grape fruit, orange, lime, menthol, liquorice, caramel aroma, honey aroma, peanut, walnut, cashew, hazelnut, almonds, pineapple, strawberry, raspberry, tropical fruits, cherries, cinnamon, peppermint, wintergreen, spearmint, eucalyptus, and mint, fruit essence such as from apple, pear, peach, strawberry, apricot, raspberry, cherry, pineapple, and plum essence. The essential oils include peppermint, spearmint, menthol, eucalyptus, clove oil, bay oil, anise, thyme, cedar leaf oil, nutmeg, and oils of the fruits mentioned above.

In various embodiments of the invention, the pouch composition comprises composition modifier. The composition modifier may be added to engineer the properties of the pouch composition and/or parts thereof, such as flowability, texture, homogeneity etc.

The composition modifiers may, according to various embodiments, be selected group consisting of metallic stearates, modified calcium carbonate, hydrogenated vegetable oils, partially hydrogenated vegetable oils, polyethylene glycols, polyoxyethylene monostearates, animal fats, silicates, silicates dioxide, talc, magnesium stearates, calcium stearates, fumed silica, powdered hydrogenated cottonseed oils, hydrogenated vegetable oils, hydrogenated soya oil, emulsifiers, triglycerides, and mixtures thereof. Particularly, metallic stearates, such as magnesium stearate, may be advantageous.

The composition modifiers may be added to the pouch composition in various ways.

For example, the composition modifiers may be added by full powder mixture during the last few minutes of the final mixing.

Alternatively, the composition modifiers may be added after granulation steps on a granulation premix.

The composition modifier, such as magnesium stearate, may have a sealing effect and can be used to control the release of the nicotine and the solubility of the pouch.

According to an embodiment of the invention, the pouch composition comprises polyvinylpyrrolidone (PVP). The pouch composition may also be free of PVP.

One advantage of the above embodiment may be that a more uniform composition may be obtained.

### EXAMPLES

### Example 1A—Preparation of Pouches Designed for Administration of Nicotine

The material of the pouches is heat sealable non-woven cellulose, such as long fiber paper. Pouches that are not in form of non-woven cellulose fabric may also be used according to the invention.

The powder is filled into pouches and is maintained in the pouch by a sealing.

### Example 1B—Preparation of Pouches Designed for Administration of Nicotine

The material of the pouches is manufactured using rayon fibers, such as viscose rayon staple fibers. The pouch membrane is heat sealed along its edges except for an opening in one end into an inner cavity formed by the pouch membrane.

The powder is filled into pouches and is maintained in the pouch by a sealing.

### Example 2: Preparation of Nicotine Premixes

A 60 liter planetary Bear Varimixer mixer was charged with water, and nicotine was weighed and added. The mixer was stirred at low speed for 1 minute at ambient temperature. Then ion exchange resin Amberlite® IRP64 was weighed and added to the mixer. The mixer was closed, stirred at high speed for 5 minutes, opened and scraped down, if necessary. Finally the mixer was stirred for further 5 minutes at high speed. The total process time was 20 minutes.

Thereby, mixtures of nicotine and cation exchange resin were produced from the constituents stated in the below tables.

Premix I:

TABLE 1
Ingredients used to manufacture nicotine premix I (5.7% nicotine).
% water in obtained nicotine-resin composition: 71.4

| Constituent | Amount (kg) | Amount (%) |
|---|---|---|
| Nicotine | 1.0 | 5.7 |
| Water | 12.5 | 71.4 |
| Resin | 4.0 | 22.9 |
| Total | 17.5 | 100.0 |

Premix II:

TABLE 2
Ingredients used to manufacture nicotine premix II (13.2% nicotine).
% water in obtained nicotine-resin composition: 34.1.

| Constituent | Amount (kg) | Amount (%) |
|---|---|---|
| Nicotine | 1.08 | 13.2 |
| Water | 2.80 | 34.1 |
| Resin | 4.32 | 52.7 |
| Total | 8.20 | 100.0 |

Premix III:

TABLE 3
Ingredients used to manufacture nicotine premix III (18.5% nicotine).
% water in obtained nicotine-resin composition: 7.5.

| Constituent | Amount (kg) | Amount (%) |
|---|---|---|
| Nicotine | 1.08 | 18.5 |
| Water | 0.44 | 7.5 |
| Resin | 4.32 | 74.0 |
| Total | 5.84 | 100.0 |

Premix IV:

TABLE 4
Ingredients used to manufacture nicotine premix IV (10% nicotine).
% water in obtained nicotine-resin composition: 50.0.

| Constituent | Amount (kg) | Amount (%) |
|---|---|---|
| Nicotine | 1.08 | 10.0 |
| Water | 5.40 | 50.0 |
| Resin | 4.32 | 40.0 |
| Total | 10.8 | 100.0 |

Premix V:

TABLE 5
Ingredients used to manufacture nicotine premix V (20% nicotine).
% water in obtained nicotine-resin composition: 31.5.

| Constituent | Amount (kg) | Amount (%) |
|---|---|---|
| Nicotine | 1.78 | 20.0 |
| Water | 2.80 | 31.5 |
| Resin | 4.32 | 48.5 |
| Total | 8.90 | 100.0 |

Premix VI:

TABLE 6
Ingredients used to manufacture nicotine premix VI (30% nicotine).
% water in obtained nicotine-resin composition: 27.5.

| Constituent | Amount (kg) | Amount (%) |
|---|---|---|
| Nicotine | 3.05 | 30.0 |
| Water | 2.80 | 27.5 |
| Resin | 4.32 | 42.5 |
| Total | 10.17 | 100.0 |

Premix VII

TABLE 7
Ingredients used to manufacture nicotine premix VII (35% nicotine).
% water in obtained nicotine-resin composition: 25.6.

| Constituent | Amount (kg) | Amount (%) |
|---|---|---|
| Nicotine | 3.83 | 35.0 |
| Water | 2.80 | 25.6 |
| Resin | 4.32 | 39.4 |
| Total | 10.95 | 100.0 |

Premix VIII:

TABLE 8
Ingredients used to manufacture nicotine premix VIII (42% nicotine).
% water in obtained nicotine-resin composition: 22.8.

| Constituent | Amount (kg) | Amount (%) |
|---|---|---|
| Nicotine | 5.15 | 42.0 |
| Water | 2.80 | 22.8 |
| Resin | 4.32 | 35.2 |
| Total | 12.27 | 100.0 |

### Example 3: Preparation of Pouch Compositions

Pouches are prepared comprising powdered compositions as outlined in table 9-21. The pouches are made as follows.

Fibers and water are mixed using a planetary Bear Varimixer mixer for 5 minutes. Then, the following ingredients were added subsequently under continuous mixing: first the nicotine-ion exchange combination (NPR or premix) (mixed for 2 minutes), then the remaining ingredients except liquid flavor and glidant if any (mixed for 2 minutes), then liquid flavor if any (mixed for 1 minute), then glidant if any (mixed for 1 minute). The total mixing time is 9-11 minutes.

### Example 4: Preparation of Filled Pouches

The final pouch composition is filled into pouches (target fill weight 500 mg powder per pouch). The pouch material of example 1A or 1B may be used. The powder is filled into pouches and is maintained in the pouch by a sealing.

### Example 5A: Pouches

The pouch compositions are prepared from the ingredients in table 9 using preparation method described in example 3.

The pouch compositions are filled into pouches as described in example 4 (pouch material of examples 1A was used, but 1B could also have been applied).

TABLE 9
Pouch compositions.

| Pouches | P01 | P02 | P03 | P04 | P05 | P06 | P07 | P08 | C1 |
|---|---|---|---|---|---|---|---|---|---|
| Amount of | 9.6 mg | 9.6 mg | 9.6 mg | 9.6 mg | 9.6 mg | 9.6 mg | 9.6 mg | 9.6 mg | 9.6 mg |
| nicotine | | | | | | | | | |
| Water | 25 | 25 | 25 | 25 | 25 | 25 | 25 | 25 | 25 |
| content | | | | | | | | | |
| [wt %] | | | | | | | | | |
| Inorganic | 0.5 | 0.75 | 1 | 1.5 | 2 | 3 | 4 | 7.5 | — |
| divalent | | | | | | | | | |
| cations | | | | | | | | | |
| [eq]* | | | | | | | | | |
| Raw material | Content in weight percent | | | | | | | | |
| NPR (16%) | 12.1 | 12.1 | 12.1 | 12.1 | 12.1 | 12.1 | 12.1 | 12.1 | 12.1 |
| CaCl₂** | 0.7 | 1.0 | 1.3 | 2.0 | 2.6 | 3.9 | 5.2 | 10.0 | — |
| Xylitol | 18.2 | 17.9 | 17.6 | 16.9 | 16.3 | 15.0 | 13.7 | 8.9 | 18.9 |
| Purified | 25 | 25 | 25 | 25 | 25 | 25 | 25 | 25 | 25 |
| water | | | | | | | | | |
| Wheat | 25 | 25 | 25 | 25 | 25 | 25 | 25 | 25 | 25 |
| fiber | | | | | | | | | |
| Sodium | 2.0 | 2.0 | 2.0 | 2.0 | 2.0 | 2.0 | 2.0 | 2.0 | 2.0 |
| alginate | | | | | | | | | |
| Sodium | 5.0 | 5.0 | 5.0 | 5.0 | 5.0 | 5.0 | 5.0 | 5.0 | 5.0 |
| carbonate | | | | | | | | | |
| Flavor | 8.9 | 8.9 | 8.9 | 8.9 | 8.9 | 8.9 | 8.9 | 8.9 | 8.9 |
| High | 1.0 | 1.0 | 1.0 | 1.0 | 1.0 | 1.0 | 1.0 | 1.0 | 1.0 |
| intensity | | | | | | | | | |
| sweetener | | | | | | | | | |
| Potassium | 0.1 | 0.1 | 0.1 | 0.1 | 0.1 | 0.1 | 0.1 | 0.1 | 0.1 |
| sorbate | | | | | | | | | |
| Silicon | 2.0 | 2.0 | 2.0 | 2.0 | 2.0 | 2.0 | 2.0 | 2.0 | 2.0 |
| dioxide | | | | | | | | | |
| Total | 100 | 100 | 100 | 100 | 100 | 100 | 100 | 100 | 100 |

*The inorganic divalent cations are presented as equivalents relative to nicotine in nicotine ion-exchange combination.
**Divalent cations may be provided as a hydrated salt, such as dihydrate, tetrahydrate, hexahydrate etc. The weight % in the table are based on the non-hydrated salt.

Pouch content: 500 mg total, i.e. nicotine conc 19.2 mg/g.

Wheat fiber, trade name “Vitacel 600 WF plus”. Other fibers may be used as well, such as water-insoluble plant fibers, such as oat fibers, pea fibers, rice fiber, maize fibers, oat fibers, tomato fibers, barley fibers, rye fibers, sugar beet fibers, buckwheat fibers, potato fibers, cellulose fibers, apple fibers, cocoa fibers, powdered cellulose, bran fibers, bamboo fibers, and cellulose fiber.

Sodium alginate, glycerol and hydroxypropyl cellulose (HPC) may be used as humectants. Other humectants as described herein may also be used in combination with sodium alginate, glycerol or HPC or as an alternative.

Sodium carbonate is used as an alkaline buffering agent. Other buffering agents as described herein may also be used in combination with sodium carbonate or an alternative.

Flavor example, a mixture of e.g. menthol and peppermint may be used. Of course, other flavors as described herein may be use as well, in combination with menthol and/or peppermint or replacing these. The flavor may be liquid or flavored or a combination, i.e. a liquid flavor and a powdered flavor is added.

Acesulfame potassium and/or sucralose may as an example be used as high intensity sweeteners. Other usable high intensity sweeteners described herein may be used in combination with or instead of acesulfame potassium and/or sucralose.

Potassium sorbate is used as a preservative. Other preservatives as described herein may also be used in combination with or instead of potassium sorbate.

Silicon dioxide is used as a glidant. Other possible glidants include e.g. magnesium stearate, starch and talc.

### Example 5B

The pouch compositions are prepared from the ingredients in table 10 using preparation method described in example 3.

The pouch compositions are filled into pouches as described in example 4 (pouch material of examples 1A was used, but 1B could also have been applied).

TABLE 10
Pouch compositions.

| Pouches | P11 | P12 | P13 | P14 | P15 | P16 | P17 | P18 | C2 | C3 |
|---|---|---|---|---|---|---|---|---|---|---|
| Amount of | 9.6 mg | 9.6 mg | 9.6 mg | 9.6 mg | 9.6 mg | 9.6 mg | 9.6 mg | 9.6 mg | 9.6 mg | 9.6 mg |
| nicotine | | | | | | | | | | |
| Water content | 15 | 25 | 30 | 35 | 30 | 30 | 30 | 10 | 25 | 25 |
| [wt %] | | | | | | | | | | |
| Inorganic | 1.0 | 1.0 | 1.0 | 1.0 | 1.0 | 1.5 | 2.0 | 1.0 | — | — |
| divalent | | | | | | | | | | |
| cations [eq]* | | | | | | | | | | |
| Raw material | Content in weight percent | | | | | | | | | |
| NPR (16%) | 12.1 | 12.1 | 12.1 | 12.1 | 12.1 | 12.1 | 12.1 | 12.1 | 12.1 | 12.1 |
| CaCl₂** | 1.3 | 1.3 | 1.3 | 1.3 | — | — | — | 1.3 | — | — |
| MgCl₂** | — | — | — | — | 1.1 | 1.7 | 2.2 | — | — | — |
| Xylitol | 37.6 | 17.6 | 7.6 | 2.6 | 12.8 | 12.2 | 11.7 | 32.6 | 12.0 | 18.2 |
| Purified water | 15 | 25 | 30 | 35 | 30 | 30 | 30 | 10 | 25 | 25 |
| Wheat fiber | 15 | 25 | 30 | 30 | 25 | 25 | 25 | 25 | 25 | 25 |
| Sodium | 2.0 | 2.0 | 2.0 | 2.0 | 2.0 | 2.0 | 2.0 | 2.0 | 2.0 | 2.0 |
| alginate | | | | | | | | | | |
| Sodium | 5.0 | 5.0 | 5.0 | 5.0 | 5.0 | 5.0 | 5.0 | 5.0 | 5.0 | 5.0 |
| carbonate | | | | | | | | | | |
| Flavor | 8.9 | 8.9 | 8.9 | 8.9 | 8.9 | 8.9 | 8.9 | 8.9 | 8.9 | 8.9 |
| NaCl | — | — | — | — | — | — | — | — | 6.9**** | 0.7*** |
| High intensity | 1.0 | 1.0 | 1.0 | 1.0 | 1.0 | 1.0 | 1.0 | 1.0 | 1.0 | 1.0 |
| sweetener | | | | | | | | | | |
| Potassium | 0.1 | 0.1 | 0.1 | 0.1 | 0.1 | 0.1 | 0.1 | 0.1 | 0.1 | 0.1 |
| sorbate | | | | | | | | | | |
| Silicon | 2.0 | 2.0 | 2.0 | 2.0 | 2.0 | 2.0 | 2.0 | 2.0 | 2.0 | 2.0 |
| dioxide | | | | | | | | | | |
| Total | 100 | 100 | 100 | 100 | 100 | 100 | 100 | 100 | 100 | 100 |

*The inorganic divalent cations are presented as equivalents relative to nicotine in nicotine ion-exchange combination.
**Divalent cations may be provided as a hydrated salt, such as dihydrate, tetrahydrate, hexahydrate etc. The weight % in the table are based on the non-hydrated salt.
***Corresponds to 1 eq of NaCl relative to nicotine in nicotine ion-exchange combination.
****Corresponds to 10 eq of NaCl relative to nicotine in nicotine ion-exchange combination.

Pouch content: 500 mg total, i.e. nicotine conc 19.2 mg/g.

Wheat fiber, trade name “Vitacel 600 WF plus”. Other fibers may be used as well, such as water-insoluble plant fibers, such as oat fibers, pea fibers, rice fiber, maize fibers, oat fibers, tomato fibers, barley fibers, rye fibers, sugar beet fibers, buckwheat fibers, potato fibers, cellulose fibers, apple fibers, cocoa fibers, powdered cellulose, bran fibers, bamboo fibers, and cellulose fiber.

Sodium alginate, glycerol and hydroxypropyl cellulose (HPC) may be used as humectants. Other humectants as described herein may also be used in combination with sodium alginate, glycerol or HPC or as an alternative.

Sodium carbonate is used as an alkaline buffering agent. Other buffering agents as described herein may also be used in combination with sodium carbonate or an alternative.

Flavor example, a mixture of e.g. menthol and peppermint may be used. Of course, other flavors as described herein may be use as well, in combination with menthol and/or peppermint or replacing these. The flavor may be liquid or flavored or a combination, i.e. a liquid flavor and a powdered flavor is added.

Acesulfame potassium and/or sucralose may as an example be used as high intensity sweeteners. Other usable high intensity sweeteners described herein may be used in combination with or instead of acesulfame potassium and/or sucralose.

Potassium sorbate is used as a preservative. Other preservatives as described herein may also be used in combination with or instead of potassium sorbate.

Silicon dioxide is used as a glidant. Other possible glidants include e.g. magnesium stearate, starch and talc.

### Example 5C

The pouch compositions are prepared from the ingredients in table 11 using preparation method described in example 3.

The pouch compositions are filled into pouches as described in example 4 (pouch material of examples 1A was used, but 1B could also have been applied).

TABLE 11
Pouch compositions.

| Pouches | P20 | P21 | P22 | P23 | P24 | P25 | P26 | P27 | P28 | P29 |
|---|---|---|---|---|---|---|---|---|---|---|
| Amount of | 9.6 mg | 9.6 mg | 9.6 mg | 9.6 mg | 9.6 mg | 9.6 mg | 9.6 mg | 4.8 mg | 7.2 mg | 12.0 mg |
| nicotine | | | | | | | | | | |
| Water | 28 | 28 | 28 | 28 | 28 | 28 | 28 | 28 | 28 | 28 |
| content [wt %] | | | | | | | | | | |
| Inorganic | 1.0 | 1.0 | 1.0 | 1.0 | 1.0 | 1.0 | 1.0 | 1.0 | 1.0 | 1.0 |
| cations [eq]* | | | | | | | | | | |
| Raw material | Content in weight percent | | | | | | | | | |
| NPR (16%) | 12.1 | 12.1 | 12.1 | 12.1 | 12.1 | 12.1 | 12.1 | 6.1 | 9.0 | 15.1 |
| CaCl₂** | — | — | — | — | — | — | — | 0.7 | 1.0 | 1.6 |
| Calcium | 1.9 | — | — | — | — | — | — | — | — | — |
| acetate** | | | | | | | | | | |
| Magnesium | — | 1.7 | — | — | — | — | — | — | — | — |
| acetate** | | | | | | | | | | |
| Calcium | — | — | 2.6 | — | — | — | — | — | — | — |
| lactate** | | | | | | | | | | |
| Magnesium | — | — | — | 2.4 | — | — | — | — | — | — |
| lactate** | | | | | | | | | | |
| FeCl₂** | — | — | — | — | 1.5 | — | — | — | — | — |
| ZnCl₂** | — | — | — | — | — | 1.6 | — | — | — | — |
| AlCl₃** | — | — | — | — | — | — | 1.6 | — | — | — |
| Xylitol | 11.0 | 11.2 | 10.3 | 10.5 | 11.4 | 11.3 | 11.3 | 18.2 | 15.0 | 8.3 |
| Purified | 28 | 28 | 28 | 28 | 28 | 28 | 28 | 28 | 28 | 28 |
| water | | | | | | | | | | |
| Wheat fiber | 28 | 28 | 28 | 28 | 28 | 28 | 28 | 28 | 28 | 28 |
| Sodium | 2.0 | 2.0 | 2.0 | 2.0 | 2.0 | 2.0 | 2.0 | 2.0 | 2.0 | 2.0 |
| alginate | | | | | | | | | | |
| Sodium | 5.0 | 5.0 | 5.0 | 5.0 | 5.0 | 5.0 | 5.0 | 5.0 | 5.0 | 5.0 |
| carbonate | | | | | | | | | | |
| Flavor | 8.9 | 8.9 | 8.9 | 8.9 | 8.9 | 8.9 | 8.9 | 8.9 | 8.9 | 8.9 |
| High intensity | 1.0 | 1.0 | 1.0 | 1.0 | 1.0 | 1.0 | 1.0 | 1.0 | 1.0 | 1.0 |
| sweetener | | | | | | | | | | |
| Potassium | 0.1 | 0.1 | 0.1 | 0.1 | 0.1 | 0.1 | 0.1 | 0.1 | 0.1 | 0.1 |
| sorbate | | | | | | | | | | |
| Silicon | 2.0 | 2.0 | 2.0 | 2.0 | 2.0 | 2.0 | 2.0 | 2.0 | 2.0 | 2.0 |
| dioxide | | | | | | | | | | |
| Total | 100 | 100 | 100 | 100 | 100 | 100 | 100 | 100 | 100 | 100 |

*The inorganic cations are presented as equivalents relative to nicotine in nicotine ion-exchange combination.
**Multivalent cations may be provided as a hydrated salt, such as dihydrate, tetrahydrate, hexahydrate etc. The weight % in the table are based on the non-hydrated salt.

Pouch content: 500 mg total.

Wheat fiber, trade name “Vitacel 600 WF plus”. Other fibers may be used as well, such as water-insoluble plant fibers, such as oat fibers, pea fibers, rice fiber, maize fibers, oat fibers, tomato fibers, barley fibers, rye fibers, sugar beet fibers, buckwheat fibers, potato fibers, cellulose fibers, apple fibers, cocoa fibers, powdered cellulose, bran fibers, bamboo fibers, and cellulose fiber.

Sodium alginate, glycerol and hydroxypropyl cellulose (HPC) may be used as humectants. Other humectants as described herein may also be used in combination with sodium alginate, glycerol or HPC or as an alternative.

Sodium carbonate is used as an alkaline buffering agent. Other buffering agents as described herein may also be used in combination with sodium carbonate or an alternative.

Flavor example, a mixture of e.g. menthol and peppermint may be used. Of course, other flavors as described herein may be use as well, in combination with menthol and/or peppermint or replacing these. The flavor may be liquid or flavored or a combination, i.e. a liquid flavor and a powdered flavor is added.

Acesulfame potassium and/or sucralose may as an example be used as high intensity sweeteners. Other usable high intensity sweeteners described herein may be used in combination with or instead of acesulfame potassium and/or sucralose.

Potassium sorbate is used as a preservative. Other preservatives as described herein may also be used in combination with or instead of potassium sorbate.

Silicon dioxide is used as a glidant. Other possible glidants include e.g. magnesium stearate, starch and talc.

### Example 5D

The pouch compositions are prepared from the ingredients in table 12 using preparation method described in example 3.

The pouch compositions are filled into pouches as described in example 4 (pouch material of examples 1A was used, but 1B could also have been applied).

TABLE 12
Pouch compositions.

| Pouches | P30 | P31 | P32 | P33 | P34 | P35 | P36 | P37 | P38 | P39 |
|---|---|---|---|---|---|---|---|---|---|---|
| Amount of nicotine | 9.6 mg | 9.6 mg | 9.6 mg | 9.6 mg | 9.6 mg | 9.6 mg | 9.6 mg | 4.8 mg | 7.2 mg | 12.0 mg |
| Water content | 28 | 28 | 28 | 28 | 28 | 28 | 28 | 28 | 28 | 28 |
| [wt %] | | | | | | | | | | |
| Inorganic cations | 1.0 | 1.0 | 1.0 | 1.0 | 1.0 | 1.0 | 1.0 | 1.0 | 1.0 | 1.0 |
| [eq]* | | | | | | | | | | |
| Raw material | Content in weight percent | | | | | | | | | |
| Premix VI | 6.4 | 6.4 | 6.4 | 6.4 | 6.4 | 6.4 | 6.4 | 3.2 | 4.8 | 8.0 |
| CaCl₂** | — | — | — | — | — | — | — | 0.7 | 1.0 | 1.6 |
| Calcium acetate** | 1.9 | — | — | — | — | — | — | — | — | — |
| Magnesium | — | 1.7 | — | — | — | — | — | — | — | — |
| acetate** | | | | | | | | | | |
| Calcium lactate** | — | — | 2.6 | — | — | — | — | — | — | — |
| Magnesium | — | — | — | 2.4 | — | — | — | — | — | — |
| lactate** | | | | | | | | | | |
| FeCl₂** | — | — | — | — | 1.5 | — | — | — | — | — |
| ZnCl₂** | — | — | — | — | — | 1.6 | — | — | — | — |
| AlCl₃** | — | — | — | — | — | — | 1.6 | — | — | — |
| Xylitol | 18.7 | 18.9 | 18.0 | 18.2 | 19.1 | 19.0 | 19.0 | 21.9 | 20.5 | 17.6 |
| Purified water | 26 | 26 | 26 | 26 | 26 | 26 | 26 | 27.2 | 26.7 | 25.8 |
| Wheat fiber | 28 | 28 | 28 | 28 | 28 | 28 | 28 | 28 | 28 | 28 |
| Sodium alginate | 2.0 | 2.0 | 2.0 | 2.0 | 2.0 | 2.0 | 2.0 | 2.0 | 2.0 | 2.0 |
| Sodium carbonate | 5.0 | 5.0 | 5.0 | 5.0 | 5.0 | 5.0 | 5.0 | 5.0 | 5.0 | 5.0 |
| Flavor | 8.9 | 8.9 | 8.9 | 8.9 | 8.9 | 8.9 | 8.9 | 8.9 | 8.9 | 8.9 |
| High intensity | 1.0 | 1.0 | 1.0 | 1.0 | 1.0 | 1.0 | 1.0 | 1.0 | 1.0 | 1.0 |
| sweetener | | | | | | | | | | |
| Potassium sorbate | 0.1 | 0.1 | 0.1 | 0.1 | 0.1 | 0.1 | 0.1 | 0.1 | 0.1 | 0.1 |
| Silicon dioxide | 2.0 | 2.0 | 2.0 | 2.0 | 2.0 | 2.0 | 2.0 | 2.0 | 2.0 | 2.0 |
| Total | 100 | 100 | 100 | 100 | 100 | 100 | 100 | 100 | 100 | 100 |

*The inorganic cations are presented as equivalents relative to nicotine in nicotine ion-exchange combination.
**Multivalent cations may be provided as a hydrated salt, such as dihydrate, tetrahydrate, hexahydrate etc. The weight % in the table are based on the non-hydrated salt.

Pouch content: 500 mg total.

Wheat fiber, trade name “Vitacel 600 WF plus”. Other fibers may be used as well, such as water-insoluble plant fibers, such as oat fibers, pea fibers, rice fiber, maize fibers, oat fibers, tomato fibers, barley fibers, rye fibers, sugar beet fibers, buckwheat fibers, potato fibers, cellulose fibers, apple fibers, cocoa fibers, powdered cellulose, bran fibers, bamboo fibers, and cellulose fiber.

Sodium alginate, glycerol and hydroxypropyl cellulose (HPC) may be used as humectants. Other humectants as described herein may also be used in combination with sodium alginate, glycerol or HPC or as an alternative.

Sodium carbonate is used as an alkaline buffering agent. Other buffering agents as described herein may also be used in combination with sodium carbonate or an alternative.

Flavor example, a mixture of e.g. menthol and peppermint may be used. Of course, other flavors as described herein may be use as well, in combination with menthol and/or peppermint or replacing these. The flavor may be liquid or flavored or a combination, i.e. a liquid flavor and a powdered flavor is added.

Acesulfame potassium and/or sucralose may as an example be used as high intensity sweeteners. Other usable high intensity sweeteners described herein may be used in combination with or instead of acesulfame potassium and/or sucralose.

Potassium sorbate is used as a preservative. Other preservatives as described herein may also be used in combination with or instead of potassium sorbate.

Silicon dioxide is used as a glidant. Other possible glidants include e.g. magnesium stearate, starch and talc.

### Example 5E

The pouch compositions are prepared from the ingredients in table 13 using preparation method described in example 3.

The pouch compositions are filled into pouches as described in example 4 (pouch material of examples 1A was used, but 1B could also have been applied).

TABLE 13
Pouch compositions.

| Pouches | P40 | P41 | P42 | P43 | P44 | P45 | C4 | C5 |
|---|---|---|---|---|---|---|---|---|
| Amount of nicotine | 9.6 mg | 9.6 mg | 9.6 mg | 9.6 mg | 9.6 mg | 9.6 mg | 9.6 mg | 9.6 mg |
| Water content | 30 | 30 | 30 | 30 | 30 | 30 | 30 | 30 |
| [wt %] | | | | | | | | |
| Inorganic divalent | 0.75 | 1.0 | 1.5 | 0.75 | 1.0 | 1.5 | — | — |
| cations [eq]* | | | | | | | | |
| Raw material | Content in weight percent | | | | | | | |
| Premix II | 14.6 | 14.6 | 14.6 | — | — | — | 14.6 | — |
| Premix VI | — | — | | 6.4 | 6.4 | 6.4 | — | 6.4 |
| CaCl₂** | 1.0 | 1.3 | 2.0 | 1.0 | 1.3 | 2.0 | — | — |
| Xylitol | 10.4 | 10.1 | 9.4 | 15.6 | 15.3 | 14.6 | 9.4 | 14.6 |
| Purified water | 25 | 25 | 25 | 28 | 28 | 28 | 25 | 28 |
| Wheat fiber | 30 | 30 | 30 | 30 | 30 | 30 | 30 | 30 |
| Sodium alginate | 2.0 | 2.0 | 2.0 | 2.0 | 2.0 | 2.0 | 2.0 | 2.0 |
| Sodium carbonate | 5.0 | 5.0 | 5.0 | 5.0 | 5.0 | 5.0 | 5.0 | 5.0 |
| NaCl | — | — | — | — | — | — | 2.0 | 2.0 |
| Flavor | 8.9 | 8.9 | 8.9 | 8.9 | 8.9 | 8.9 | 8.9 | 8.9 |
| High intensity | 1.0 | 1.0 | 1.0 | 1.0 | 1.0 | 1.0 | 1.0 | 1.0 |
| sweetener | | | | | | | | |
| Potassium sorbate | 0.1 | 0.1 | 0.1 | 0.1 | 0.1 | 0.1 | 0.1 | 0.1 |
| Silicon dioxide | 2.0 | 2.0 | 2.0 | 2.0 | 2.0 | 2.0 | 2.0 | 2.0 |
| Total | 100 | 100 | 100 | 100 | 100 | 100 | 100 | 100 |

*The inorganic divalent cations are presented as equivalents relative to nicotine in nicotine ion-exchange combination.
**Divalent cations may be provided as a hydrated salt, such as dihydrate, tetrahydrate, hexahydrate etc. The weight % in the table are based on the non-hydrated salt.

Pouch content: 500 mg total, i.e. nicotine conc 19.2 mg/g.

Wheat fiber, trade name “Vitacel 600 WF plus”. Other fibers may be used as well, such as water-insoluble plant fibers, such as oat fibers, pea fibers, rice fiber, maize fibers, oat fibers, tomato fibers, barley fibers, rye fibers, sugar beet fibers, buckwheat fibers, potato fibers, cellulose fibers, apple fibers, cocoa fibers, powdered cellulose, bran fibers, bamboo fibers, and cellulose fiber.

Sodium alginate, glycerol and hydroxypropyl cellulose (HPC) may be used as humectants. Other humectants as described herein may also be used in combination with sodium alginate, glycerol or HPC or as an alternative.

Sodium carbonate is used as an alkaline buffering agent. Other buffering agents as described herein may also be used in combination with sodium carbonate or an alternative.

Flavor example, a mixture of e.g. menthol and peppermint may be used. Of course, other flavors as described herein may be use as well, in combination with menthol and/or peppermint or replacing these. The flavor may be liquid or flavored or a combination, i.e. a liquid flavor and a powdered flavor is added.

Acesulfame potassium and/or sucralose may as an example be used as high intensity sweeteners. Other usable high intensity sweeteners described herein may be used in combination with or instead of acesulfame potassium and/or sucralose.

Potassium sorbate is used as a preservative. Other preservatives as described herein may also be used in combination with or instead of potassium sorbate.

Silicon dioxide is used as a glidant. Other possible glidants include e.g. magnesium stearate, starch and talc.

### Example 5F

The pouch compositions are prepared from the ingredients in table 14 using preparation method described in example 3.

The pouch compositions are filled into pouches as described in example 4 (pouch material of examples 1A was used, but 1B could also have been applied).

TABLE 14
Pouch compositions.

| Pouches | P50 | P51 | P52 | P53 | P54 | P55 | C6 | C7 |
|---|---|---|---|---|---|---|---|---|
| Amount of | 9.6 mg | 9.6 mg | 9.6 mg | 9.6 mg | 9.6 mg | 9.6 mg | 9.6 mg | 9.6 mg |
| nicotine | | | | | | | | |
| Water content | 15 | 25 | 40 | 30 | 30 | 10 | 30 | 30 |
| [wt %] | | | | | | | | |
| Inorganic divalent | 1.0 | 1.0 | 1.0 | 1.0 | 1.0 | 1.0 | — | — |
| cations [eq]* | | | | | | | | |
| Raw material | Content in weight percent | | | | | | | |
| Premix VI | 6.4 | 6.4 | 6.4 | — | — | 6.4 | — | — |
| Premix VII | — | — | — | 5.5 | — | — | 5.5 | — |
| Premix VIII | — | — | — | — | 4.6 | — | | 4.6 |
| CaCl₂** | 1.3 | 1.3 | 1.3 | 1.3 | 1.3 | 1.3 | — | — |
| Xylitol | 45.3 | 25.3 | 4.3 | 15.6 | 16.1 | 49.3 | 21.9 | 17.4 |
| Purified water | 13 | 23 | 38 | 28.6 | 29 | 9 | 28.6 | 29 |
| Wheat fiber | 15 | 25 | 31 | 30 | 30 | 15 | 25 | 30 |
| Sodium alginate | 2.0 | 2.0 | 2.0 | 2.0 | 2.0 | 2.0 | 2.0 | 2.0 |
| Sodium | 5.0 | 5.0 | 5.0 | 5.0 | 5.0 | 5.0 | 5.0 | 5.0 |
| carbonate | | | | | | | | |
| Flavor | 8.9 | 8.9 | 8.9 | 8.9 | 8.9 | 8.9 | 8.9 | 8.9 |
| High intensity | 1.0 | 1.0 | 1.0 | 1.0 | 1.0 | 1.0 | 1.0 | 1.0 |
| sweetener | | | | | | | | |
| Potassium | 0.1 | 0.1 | 0.1 | 0.1 | 0.1 | 0.1 | 0.1 | 0.1 |
| sorbate | | | | | | | | |
| Silicon dioxide | 2.0 | 2.0 | 2.0 | 2.0 | 2.0 | 2.0 | 2.0 | 2.0 |
| Total | 100 | 100 | 100 | 100 | 100 | 100 | 100 | 100 |

*The inorganic divalent cations are presented as equivalents relative to nicotine in nicotine ion-exchange combination.
**Divalent cations may be provided as a hydrated salt, such as dihydrate, tetrahydrate, hexahydrate etc. The weight % in the table are based on the non- hydrated salt.

Pouch content: 500 mg total, i.e. nicotine conc 19.2 mg/g.

Wheat fiber, trade name “Vitacel 600 WF plus”. Other fibers may be used as well, such as water-insoluble plant fibers, such as oat fibers, pea fibers, rice fiber, maize fibers, oat fibers, tomato fibers, barley fibers, rye fibers, sugar beet fibers, buckwheat fibers, potato fibers, cellulose fibers, apple fibers, cocoa fibers, powdered cellulose, bran fibers, bamboo fibers, and cellulose fiber.

Sodium alginate, glycerol and hydroxypropyl cellulose (HPC) may be used as humectants. Other humectants as described herein may also be used in combination with sodium alginate, glycerol or HPC or as an alternative.

Sodium carbonate is used as an alkaline buffering agent. Other buffering agents as described herein may also be used in combination with sodium carbonate or an alternative.

Flavor example, a mixture of e.g. menthol and peppermint may be used. Of course, other flavors as described herein may be use as well, in combination with menthol and/or peppermint or replacing these. The flavor may be liquid or flavored or a combination, i.e. a liquid flavor and a powdered flavor is added.

Acesulfame potassium and/or sucralose may as an example be used as high intensity sweeteners. Other usable high intensity sweeteners described herein may be used in combination with or instead of acesulfame potassium and/or sucralose.

Potassium sorbate is used as a preservative. Other preservatives as described herein may also be used in combination with or instead of potassium sorbate.

Silicon dioxide is used as a glidant. Other possible glidants include e.g. magnesium stearate, starch and talc.

### Example 5G

The pouch compositions are prepared from the ingredients in table 15 using preparation method described in example 3.

The pouch compositions are filled into pouches as described in example 4 (pouch material of examples 1A was used, but 1B could also have been applied).

TABLE 15
Pouch compositions.

| Pouches | P60 | P61 | P62 | P63 | P64 | P65 | P66 | P67 | C8 | C9 |
|---|---|---|---|---|---|---|---|---|---|---|
| Amount of | 9.6 mg | 9.6 mg | 9.6 mg | 9.6 mg | 9.6 mg | 9.6 mg | 9.6 mg | 9.6 mg | 9.6 mg | 9.6 mg |
| nicotine | | | | | | | | | | |
| Water content | 27 | 27 | 27 | 27 | 27 | 27 | 35 | 30 | 30 | 30 |
| [wt %] | | | | | | | | | | |
| Inorganic divalent | 1.0 | 1.0 | 1.0 | 1.0 | 1.0 | 1.0 | 1.0 | 1.0 | — | — |
| cations [eq]* | | | | | | | | | | |
| Raw material | Content in weight percent | | | | | | | | | |
| Premix VI | 6.4 | 6.4 | 6.4 | 6.4 | 6.4 | 6.4 | 6.4 | 6.4 | 6.4 | 6.4 |
| CaCl₂** | 1.3 | 1.3 | 1.3 | 1.3 | 1.3 | 1.3 | 1.3 | 1.3 | — | — |
| Xylitol | 5.0 | — | — | — | — | 7.0 | | 5.0 | 5.0 | 5.0 |
| Isomalt | — | 21.3 | — | — | — | — | | — | — | — |
| Sorbitol | — | — | 21.3 | — | — | — | | — | — | — |
| Mannitol | — | — | — | 21.3 | — | — | | — | — | — |
| Maltitol | — | — | — | — | 21.3 | — | | — | — | — |
| Erythritol | 16.3 | — | — | — | — | 14.3 | | 14.2 | 23.5 | 18.5 |
| Purified water | 25 | 25 | 25 | 25 | 25 | 25 | 33 | 28 | 28 | 28 |
| Wheat fiber | 27 | 27 | 27 | 27 | 27 | 27 | 40.3 | 30 | 15 | 15 |
| Sodium alginate | 2.0 | 2.0 | 2.0 | 2.0 | 2.0 | 2.0 | 2.0 | 2.0 | 2.0 | 2.0 |
| Sodium | 5.0 | 5.0 | 5.0 | 5.0 | 5.0 | 5.0 | 5.0 | 5.0 | 5.0 | 5.0 |
| carbonate | | | | | | | | | | |
| Flavor | 8.9 | 8.9 | 8.9 | 8.9 | 8.9 | 8.9 | 8.9 | 5.0 | 7.0 | 7.0 |
| NaCl | — | — | — | — | — | — | — | — | 5.0 | 10.0 |
| High intensity | 1.0 | 1.0 | 1.0 | 1.0 | 1.0 | 1.0 | 1.0 | 1.0 | 1.0 | 1.0 |
| sweetener | | | | | | | | | | |
| Potassium | 0.1 | 0.1 | 0.1 | 0.1 | 0.1 | 0.1 | 0.1 | 0.1 | 0.1 | 0.1 |
| sorbate | | | | | | | | | | |
| Silicon dioxide | 2.0 | 2.0 | 2.0 | 2.0 | 2.0 | 2.0 | 2.0 | 2.0 | 2.0 | 2.0 |
| Total | 100 | 100 | 100 | 100 | 100 | 100 | 100 | 100 | 100 | 100 |

*The inorganic divalent cations are presented as equivalents relative to nicotine in nicotine ion-exchange combination.
**Divalent cations may be provided as a hydrated salt, such as dihydrate, tetrahydrate, hexahydrate etc. The weight % in the table are based on the non-hydrated salt.

Pouch content: 500 mg total, i.e. nicotine conc 19.2 mg/g.

Wheat fiber, trade name “Vitacel 600 WF plus”. Other fibers may be used as well, such as water-insoluble plant fibers, such as oat fibers, pea fibers, rice fiber, maize fibers, oat fibers, tomato fibers, barley fibers, rye fibers, sugar beet fibers, buckwheat fibers, potato fibers, cellulose fibers, apple fibers, cocoa fibers, powdered cellulose, bran fibers, bamboo fibers, and cellulose fiber.

Sodium alginate, glycerol and hydroxypropyl cellulose (HPC) may be used as humectants. Other humectants as described herein may also be used in combination with sodium alginate, glycerol or HPC or as an alternative.

Sodium carbonate is used as an alkaline buffering agent. Other buffering agents as described herein may also be used in combination with sodium carbonate or an alternative.

Flavor example, a mixture of e.g. menthol and peppermint may be used. Of course, other flavors as described herein may be use as well, in combination with menthol and/or peppermint or replacing these. The flavor may be liquid or flavored or a combination, i.e. a liquid flavor and a powdered flavor is added.

Acesulfame potassium and/or sucralose may as an example be used as high intensity sweeteners. Other usable high intensity sweeteners described herein may be used in combination with or instead of acesulfame potassium and/or sucralose.

Potassium sorbate is used as a preservative. Other preservatives as described herein may also be used in combination with or instead of potassium sorbate.

Silicon dioxide is used as a glidant. Other possible glidants include e.g. magnesium stearate, starch and talc.

### Example 5H

The pouch compositions are prepared from the ingredients in table 16 using preparation method described in example 3.

The pouch compositions are filled into pouches as described in example 4 (pouch material of examples 1A was used, but 1B could also have been applied).

TABLE 16
Pouch compositions.

| Pouches | P70 | P71 | P72 | P73 | P74 | P75 | P76 | P77 |
|---|---|---|---|---|---|---|---|---|
| Amount of nicotine | 9.6 mg | 9.6 mg | 9.6 mg | 9.6 mg | 9.6 mg | 9.6 mg | 9.6 mg | 9.6 mg |
| Water content [wt %] | 27 | 27 | 27 | 20 | 20 | 35 | 20 | 35 |
| Inorganic divalent cations | 1.0 | 1.0 | 1.0 | 1.0 | 1.0 | 1.0 | 1.0 | 1.0 |
| [eq]* | | | | | | | | |
| Raw material | Content in weight percent | | | | | | | |
| Premix VI | 6.4 | 6.4 | 6.4 | 6.4 | 6.4 | 6.4 | 6.4 | 6.4 |
| CaCl₂** | 1.3 | 1.3 | 1.3 | 1.3 | 1.3 | 1.3 | 1.3 | 1.3 |
| Xylitol | 5.0 | 5.0 | 5.0 | 5.0 | 5.0 | 5.0 | 5.0 | 5.0 |
| Erythritol | 18.2 | 18.2 | 18.2 | 42.2 | 22.2 | 17.2 | 22.2 | 17.2 |
| Purified water | 25 | 25 | 25 | 18 | 18 | 33 | 18 | 33 |
| Wheat fiber | — | — | — | 10 | 30 | 20 | — | — |
| Oat fiber | 27 | — | — | — | — | — | 30 | 20 |
| Pea Fiber | — | 27 | — | — | — | — | — | — |
| Powdered Cellulose | — | — | 27 | — | — | — | — | — |
| Sodium alginate | 2.0 | 2.0 | 2.0 | 2.0 | 2.0 | 2.0 | 2.0 | 2.0 |
| Sodium carbonate | 5.0 | 5.0 | 5.0 | 5.0 | 5.0 | 5.0 | 5.0 | 5.0 |
| Flavor | 7.0 | 7.0 | 7.0 | 7.0 | 7.0 | 7.0 | 7.0 | 7.0 |
| High intensity sweetener | 1.0 | 1.0 | 1.0 | 1.0 | 1.0 | 1.0 | 1.0 | 1.0 |
| Potassium sorbate | 0.1 | 0.1 | 0.1 | 0.1 | 0.1 | 0.1 | 0.1 | 0.1 |
| Silicon dioxide | 2.0 | 2.0 | 2.0 | 2.0 | 2.0 | 2.0 | 2.0 | 2.0 |
| Total | 100 | 100 | 100 | 100 | 100 | 100 | 100 | 100 |

*The inorganic divalent cations are presented as equivalents relative to nicotine in nicotine ion-exchange combination.
**Divalent cations may be provided as a hydrated salt, such as dihydrate, tetrahydrate, hexahydrate etc. The weight % in the table are based on the non- hydrated salt.

Pouch content: 500 mg total, i.e. nicotine conc 19.2 mg/g.

Wheat fiber, trade name “Vitacel 600 WF plus” or “Vitacel 200WF”.

Powdered cellulose, trade name “Vitacel L00” or “Vitacel L700G”.

Oat fiber, trade name “Vitacel HF 600”.

Pea fiber, trade name “Vitacel EF150”.

Other fibers may be used as well, such as water-insoluble plant fibers, such as oat fibers, pea fibers, rice fiber, maize fibers, oat fibers, tomato fibers, barley fibers, rye fibers, sugar beet fibers, buckwheat fibers, potato fibers, powdered cellulose, cellulose fibers, apple fibers, cocoa fibers, bamboo fibers, bran fibers, and cellulose fiber.

Sodium alginate, glycerol and hydroxypropyl cellulose (HPC) may be used as humectants. Other humectants as described herein may also be used in combination with sodium alginate, glycerol or HPC or as an alternative.

Sodium carbonate is used as an alkaline buffering agent. Other buffering agents as described herein may also be used in combination with sodium carbonate or an alternative.

Flavor example, a mixture of e.g. menthol and peppermint may be used. Of course, other flavors as described herein may be use as well, in combination with menthol and/or peppermint or replacing these. The flavor may be liquid or flavored or a combination, i.e. a liquid flavor and a powdered flavor is added.

Acesulfame potassium and/or sucralose may as an example be used as high intensity sweeteners. Other usable high intensity sweeteners described herein may be used in combination with or instead of acesulfame potassium and/or sucralose.

Potassium sorbate is used as a preservative. Other preservatives as described herein may also be used in combination with or instead of potassium sorbate.

Silicon dioxide is used as a glidant. Other possible glidants include e.g. magnesium stearate, starch and talc.

### Example 5I

The pouch compositions are prepared from the ingredients in table 17 using preparation method described in example 3.

The pouch compositions are filled into pouches as described in example 4 (pouch material of examples 1A was used, but 1B could also have been applied).

TABLE 17
Pouch compositions.

| Pouches | P80 | P81 | P82 | P83 | P84 | P85 | P86 | P87 | P88 |
|---|---|---|---|---|---|---|---|---|---|
| Amount of | 9.6 mg | 9.6 mg | 9.6 mg | 9.6 mg | 9.6 mg | 9.6 mg | 9.6 mg | 9.6 mg | 9.6 mg |
| nicotine | | | | | | | | | |
| Water content | 28 | 28 | 28 | 28 | 28 | 35 | 28 | 28 | 28 |
| [wt %] | | | | | | | | | |
| Inorganic divalent | 1.0 | 1.0 | 1.0 | 1.0 | 1.0 | 1.0 | 1.0 | 1.0 | — |
| cations [eq]* | | | | | | | | | |
| Raw material | Content in weight percent | | | | | | | | |
| NPR (16%) | 12.1 | 12.1 | 12.1 | 12.1 | 12.1 | 12.1 | 12.1 | 12.1 | 12.1 |
| CaCl₂** | 1.3 | 1.3 | 1.3 | 1.3 | 1.3 | 1.3 | 1.3 | 1.3 | 1.3 |
| Xylitol | 5.0 | — | — | — | — | — | 5.0 | 5.0 | 5.0 |
| Isomalt | — | 13.5 | — | — | — | — | — | — | — |
| Sorbitol | — | — | 13.5 | — | — | — | — | — | — |
| Mannitol | — | — | — | 13.5 | — | — | — | — | — |
| Maltitol | — | — | — | — | 13.5 | — | — | — | — |
| Erythritol | 8.5 | — | — | — | — | — | 8.5 | 8.5 | 8.5 |
| Purified water | 28 | 28 | 28 | 28 | 28 | 35 | 28 | 28 | 28 |
| Wheat fiber | 28 | 28 | 28 | 28 | 28 | 34.5 | — | — | — |
| Oat fiber | — | — | — | — | — | — | 28 | — | — |
| Pea Fiber | — | — | — | — | — | — | — | 28 | — |
| Powdered | — | — | — | — | — | — | — | — | 28 |
| Cellulose | | | | | | | | | |
| Sodium alginate | 2.0 | 2.0 | 2.0 | 2.0 | 2.0 | 2.0 | 2.0 | 2.0 | 2.0 |
| Sodium | 5.0 | 5.0 | 5.0 | 5.0 | 5.0 | 5.0 | 5.0 | 5.0 | 5.0 |
| carbonate | | | | | | | | | |
| Flavor | 7.0 | 7.0 | 7.0 | 7.0 | 7.0 | 7.0 | 7.0 | 7.0 | 7.0 |
| High intensity | 1.0 | 1.0 | 1.0 | 1.0 | 1.0 | 1.0 | 1.0 | 1.0 | 1.0 |
| sweetener | | | | | | | | | |
| Potassium | 0.1 | 0.1 | 0.1 | 0.1 | 0.1 | 0.1 | 0.1 | 0.1 | 0.1 |
| sorbate | | | | | | | | | |
| Silicon dioxide | 2.0 | 2.0 | 2.0 | 2.0 | 2.0 | 2.0 | 2.0 | 2.0 | 2.0 |
| Total | 100 | 100 | 100 | 100 | 100 | 100 | 100 | 100 | 100 |

*The inorganic divalent cations are presented as equivalents relative to nicotine in nicotine ion-exchange combination.
**Divalent cations may be provided as a hydrated salt, such as dihydrate, tetrahydrate, hexahydrate etc. The weight % in the table are based on the non-hydrated salt.

Pouch content: 500 mg total, i.e. nicotine conc 19.2 mg/g.

Wheat fiber, trade name “Vitacel 600 WF plus” or “Vitacel 200WF”.

Powdered cellulose, trade name “Vitacel L00” or “Vitacel L700G”.

Oat fiber, trade name “Vitacel HF 600”.

Pea fiber, trade name “Vitacel EF150”.

Other fibers may be used as well, such as water-insoluble plant fibers, such as oat fibers, pea fibers, rice fiber, maize fibers, oat fibers, tomato fibers, barley fibers, rye fibers, sugar beet fibers, buckwheat fibers, potato fibers, powdered cellulose, cellulose fibers, apple fibers, cocoa fibers, bamboo fibers, bran fibers, and cellulose fiber.

Sodium alginate, glycerol and hydroxypropyl cellulose (HPC) may be used as humectants. Other humectants as described herein may also be used in combination with sodium alginate, glycerol or HPC or as an alternative.

Sodium carbonate is used as an alkaline buffering agent. Other buffering agents as described herein may also be used in combination with sodium carbonate or an alternative.

Flavor example, a mixture of e.g. menthol and peppermint may be used. Of course, other flavors as described herein may be use as well, in combination with menthol and/or peppermint or replacing these. The flavor may be liquid or flavored or a combination, i.e. a liquid flavor and a powdered flavor is added.

Acesulfame potassium and/or sucralose may as an example be used as high intensity sweeteners. Other usable high intensity sweeteners described herein may be used in combination with or instead of acesulfame potassium and/or sucralose.

Potassium sorbate is used as a preservative. Other preservatives as described herein may also be used in combination with or instead of potassium sorbate.

Silicon dioxide is used as a glidant. Other possible glidants include e.g. magnesium stearate, starch and talc.

### Example 5J

The pouch compositions are prepared from the ingredients in table 18 using preparation method described in example 3.

The pouch compositions are filled into pouches as described in example 4 (pouch material of examples 1A was used, but 1B could also have been applied).

TABLE 18
Pouch compositions.

| Pouches | P90 | P91 | P92 | P93 | P94 | P95 | P96 | P97 | P98 |
|---|---|---|---|---|---|---|---|---|---|
| Amount of | 9.6 mg | 9.6 mg | 9.6 mg | 9.6 mg | 9.6 mg | 9.6 mg | 9.6 mg | 9.6 mg | 9.6 mg |
| nicotine | | | | | | | | | |
| Water content | 30 | 30 | 27 | 27 | 27 | 30 | 30 | 30 | 30 |
| [wt %] | | | | | | | | | |
| Inorganic | 1.0 | 1.0 | 1.0 | 1.0 | 1.0 | 1.0 | 1.0 | 1.0 | 1.0 |
| divalent cations | | | | | | | | | |
| [eq]* | | | | | | | | | |
| Raw material | Content in weight percent | | | | | | | | |
| NPR (16%) | 7.0 | 12.1 | 12.1 | 12.1 | 12.1 | 12.1 | 12.1 | 12.1 | 12.1 |
| NBT | 2.3 | — | — | — | — | — | — | — | — |
| CaCl₂** | 1.3 | 1.3 | 1.3 | 1.3 | 1.3 | 1.3 | 1.3 | 1.3 | 1.3 |
| Xylitol | 10.4 | 10.1 | 8.6 | 11.6 | 11.6 | 12.6 | 7.6 | 7.6 | 11.5 |
| Purified water | 30 | 30 | 27 | 27 | 27 | 30 | 30 | 30 | 30 |
| Wheat fiber | 30 | 30 | 27 | 27 | 27 | 30 | 30 | 30 | 30 |
| Sodium alginate | 2.0 | 2.0 | 2.0 | 2.0 | 2.0 | 2.0 | — | — | — |
| Glycerol | — | — | — | — | — | — | 2.0 | — | — |
| Hydroxypropyl | — | — | — | — | — | — | — | 2.0 | — |
| cellulose | | | | | | | | | |
| Sodium | 5.0 | 2.5 | 10.0 | 3.5 | — | — | 5.0 | 5.0 | 5.0 |
| carbonate | | | | | | | | | |
| Sodium | — | — | — | 3.5 | — | — | — | — | — |
| hydrogen- | | | | | | | | | |
| carbonate | | | | | | | | | |
| Trometamol | — | — | — | — | 7.0 | — | — | — | — |
| Flavor | 8.9 | 8.9 | 8.9 | 8.9 | 8.9 | 8.9 | 8.9 | 8.9 | 7.0 |
| High intensity | 1.0 | 1.0 | 1.0 | 1.0 | 1.0 | 1.0 | 1.0 | 1.0 | 1.0 |
| sweetener | | | | | | | | | |
| Potassium | 0.1 | 0.1 | 0.1 | 0.1 | 0.1 | 0.1 | 0.1 | 0.1 | 0.1 |
| sorbate | | | | | | | | | |
| Silicon dioxide | 2.0 | 2.0 | 2.0 | 2.0 | 2.0 | 2.0 | 2.0 | 2.0 | 2.0 |
| Total | 100 | 100 | 100 | 100 | 100 | 100 | 100 | 100 | 100 |

*The inorganic divalent cations are presented as equivalents relative to nicotine in nicotine ion-exchange combination.
**Divalent cations may be provided as a hydrated salt, such as dihydrate, tetrahydrate, hexahydrate etc. The weight % in the table are based on the non-hydrated salt.

Pouch content: 500 mg total, i.e. nicotine conc 19.2 mg/g.

Wheat fiber, trade name “Vitacel 600 WF plus”. Other fibers may be used as well, such as water-insoluble plant fibers, such as oat fibers, pea fibers, rice fiber, maize fibers, oat fibers, tomato fibers, barley fibers, rye fibers, sugar beet fibers, buckwheat fibers, potato fibers, cellulose fibers, apple fibers, cocoa fibers, powdered cellulose, bran fibers, bamboo fibers, and cellulose fiber.

Sodium alginate, glycerol and hydroxypropyl cellulose (HPC) may be used as humectants. Other humectants as described herein may also be used in combination with sodium alginate, glycerol or HPC or as an alternative.

Sodium carbonate is used as an alkaline buffering agent. Other buffering agents as described herein may also be used in combination with sodium carbonate or an alternative.

Flavor example, a mixture of e.g. menthol and peppermint may be used. Of course, other flavors as described herein may be use as well, in combination with menthol and/or peppermint or replacing these. The flavor may be liquid or flavored or a combination, i.e. a liquid flavor and a powdered flavor is added.

Acesulfame potassium and/or sucralose may as an example be used as high intensity sweeteners. Other usable high intensity sweeteners described herein may be used in combination with or instead of acesulfame potassium and/or sucralose.

Potassium sorbate is used as a preservative. Other preservatives as described herein may also be used in combination with or instead of potassium sorbate.

Silicon dioxide is used as a glidant. Other possible glidants include e.g. magnesium stearate, starch and talc.

### Example 5K

The pouch compositions are prepared from the ingredients in table 19 using preparation method described in example 3.

The pouch compositions are filled into pouches as described in example 4 (pouch material of examples 1A was used, but 1B could also have been applied).

TABLE 19
Pouch compositions.

| Pouches | P100 | P101 | P102 | P103 | P104 | P105 | P106 | P107 | P108 |
|---|---|---|---|---|---|---|---|---|---|
| Amount of | 9.6 mg | 9.6 mg | 9.6 mg | 9.6 mg | 9.6 mg | 9.6 mg | 9.6 mg | 9.6 mg | 9.6 mg |
| nicotine | | | | | | | | | |
| Water content | 30 | 30 | 27 | 27 | 27 | 30 | 30 | 30 | 30 |
| [wt %] | | | | | | | | | |
| Inorganic divalent | 1.0 | 1.0 | 1.0 | 1.0 | 1.0 | 1.0 | 1.0 | 1.0 | 1.0 |
| cations [eq]* | | | | | | | | | |
| Raw material | Content in weight percent | | | | | | | | |
| Premix VI | 3.7 | 6.4 | 6.4 | 6.4 | 6.4 | 6.4 | 6.4 | 6.4 | 6.4 |
| NBT | 2.3 | — | — | — | — | — | — | — | — |
| CaCl₂** | 1.3 | 1.3 | 1.3 | 1.3 | 1.3 | 1.3 | 1.3 | 1.3 | 1.3 |
| Xylitol | 14.7 | 17.8 | 13.3 | 16.3 | 16.3 | 20.3 | 15.3 | 15.3 | 17.3 |
| Purified water | 29 | 28 | 25 | 25 | 25 | 28 | 28 | 28 | 28 |
| Wheat fiber | 30 | 30 | 30 | 30 | 30 | 30 | 30 | 30 | 30 |
| Sodium alginate | 2.0 | 2.0 | 2.0 | 2.0 | 2.0 | 2.0 | — | — | — |
| Glycerol | — | — | — | — | — | — | 2.0 | — | — |
| Hydroxypropyl | — | — | — | — | — | — | — | 2.0 | — |
| cellulose | | | | | | | | | |
| Sodium | 5.0 | 2.5 | 10.0 | 3.5 | — | — | 5.0 | 5.0 | 5.0 |
| carbonate | | | | | | | | | |
| Sodium | — | — | — | 3.5 | — | — | — | — | — |
| hydrogencarbonate | | | | | | | | | |
| Trometamol | — | — | — | — | 7.0 | — | — | — | — |
| Flavor | 8.9 | 8.9 | 8.9 | 8.9 | 8.9 | 8.9 | 8.9 | 8.9 | 8.9 |
| High intensity | 1.0 | 1.0 | 1.0 | 1.0 | 1.0 | 1.0 | 1.0 | 1.0 | 1.0 |
| sweetener | | | | | | | | | |
| Potassium | 0.1 | 0.1 | 0.1 | 0.1 | 0.1 | 0.1 | 0.1 | 0.1 | 0.1 |
| sorbate | | | | | | | | | |
| Silicon dioxide | 2.0 | 2.0 | 2.0 | 2.0 | 2.0 | 2.0 | 2.0 | 2.0 | 2.0 |
| Total | 100 | 100 | 100 | 100 | 100 | 100 | 100 | 100 | 100 |

*The inorganic divalent cations are presented as equivalents relative to nicotine in nicotine ion-exchange combination.
**Divalent cations may be provided as a hydrated salt, such as dihydrate, tetrahydrate, hexahydrate etc. The weight % in the table are based on the non-hydrated salt.

Pouch content: 500 mg total, i.e. nicotine conc 19.2 mg/g.

Wheat fiber, trade name “Vitacel 600 WF plus”. Other fibers may be used as well, such as water-insoluble plant fibers, such as oat fibers, pea fibers, rice fiber, maize fibers, oat fibers, tomato fibers, barley fibers, rye fibers, sugar beet fibers, buckwheat fibers, potato fibers, cellulose fibers, apple fibers, cocoa fibers, powdered cellulose, bran fibers, bamboo fibers, and cellulose fiber.

Sodium alginate, glycerol and hydroxypropyl cellulose (HPC) may be used as humectants. Other humectants as described herein may also be used in combination with sodium alginate, glycerol or HPC or as an alternative.

Sodium carbonate is used as an alkaline buffering agent. Other buffering agents as described herein may also be used in combination with sodium carbonate or an alternative.

Flavor example, a mixture of e.g. menthol and peppermint may be used. Of course, other flavors as described herein may be use as well, in combination with menthol and/or peppermint or replacing these. The flavor may be liquid or flavored or a combination, i.e. a liquid flavor and a powdered flavor is added.

Acesulfame potassium and/or sucralose may as an example be used as high intensity sweeteners. Other usable high intensity sweeteners described herein may be used in combination with or instead of acesulfame potassium and/or sucralose.

Potassium sorbate is used as a preservative. Other preservatives as described herein may also be used in combination with or instead of potassium sorbate.

Silicon dioxide is used as a glidant. Other possible glidants include e.g. magnesium stearate, starch and talc.

### Example 5L

The pouch compositions are prepared from the ingredients in table 20 using preparation method described in example 3.

The pouch compositions are filled into pouches as described in example 4 (pouch material of examples 1A was used, but 1B could also have been applied).

TABLE 20
Pouch compositions.

| Pouches | P110 | P111 | P112 | P113 | P114 | P115 | C10 | C11 |
|---|---|---|---|---|---|---|---|---|
| Amount of | 9.6 mg | 9.6 mg | 9.6 mg | 9.6 mg | 9.6 mg | 9.6 mg | 9.6 mg | 9.6 mg |
| nicotine | | | | | | | | |
| Water content | 30 | 30 | 30 | 30 | 30 | 30 | 30 | 30 |
| [wt %] | | | | | | | | |
| Inorganic divalent | 0.75 | 1.0 | 1.5 | 0.75 | 1.0 | 1.5 | — | — |
| cations [eq]* | | | | | | | | |
| Raw material | Content in weight percent | | | | | | | |
| Premix II | 14.6 | 14.6 | 14.6 | — | — | — | 14.6 | — |
| Premix VI | — | — | — | 6.4 | 6.4 | 6.4 | — | 6.4 |
| CaCl₂** | 1.0 | 1.3 | 2.0 | 1.0 | 1.3 | 2.0 | — | — |
| Xylitol | 12.4 | 12.1 | 11.4 | 17.6 | 17.3 | 16.6 | 13.4 | 18.6 |
| Purified water | 25 | 25 | 25 | 28 | 28 | 28 | 25 | 28 |
| Wheat fiber | 30 | 30 | 30 | 30 | 30 | 30 | 30 | 30 |
| Sodium | 5.0 | 5.0 | 5.0 | 5.0 | 5.0 | 5.0 | 5.0 | 5.0 |
| carbonate | | | | | | | | |
| Flavor | 8.9 | 8.9 | 8.9 | 8.9 | 8.9 | 8.9 | 8.9 | 8.9 |
| High intensity | 1.0 | 1.0 | 1.0 | 1.0 | 1.0 | 1.0 | 1.0 | 1.0 |
| sweetener | | | | | | | | |
| Potassium | 0.1 | 0.1 | 0.1 | 0.1 | 0.1 | 0.1 | 0.1 | 0.1 |
| sorbate | | | | | | | | |
| Silicon dioxide | 2.0 | 2.0 | 2.0 | 2.0 | 2.0 | 2.0 | 2.0 | 2.0 |
| Total | 100 | 100 | 100 | 100 | 100 | 100 | 100 | 100 |

*The inorganic divalent cations are presented as equivalents relative to nicotine in nicotine ion-exchange combination.
**Divalent cations may be provided as a hydrated salt, such as dihydrate, tetrahydrate, hexahydrate etc. The weight % in the table are based on the non-hydrated salt.

Pouch content: 500 mg total, i.e. nicotine conc 19.2 mg/g.

Wheat fiber, trade name “Vitacel 600 WF plus”. Other fibers may be used as well, such as water-insoluble plant fibers, such as oat fibers, pea fibers, rice fiber, maize fibers, oat fibers, tomato fibers, barley fibers, rye fibers, sugar beet fibers, buckwheat fibers, potato fibers, cellulose fibers, apple fibers, cocoa fibers, powdered cellulose, bran fibers, bamboo fibers, and cellulose fiber.

Sodium carbonate is used as an alkaline buffering agent. Other buffering agents as described herein may also be used in combination with sodium carbonate or an alternative.

Flavor example, a mixture of e.g. menthol and peppermint may be used. Of course, other flavors as described herein may be use as well, in combination with menthol and/or peppermint or replacing these. The flavor may be liquid or flavored or a combination, i.e. a liquid flavor and a powdered flavor is added.

Acesulfame potassium and/or sucralose may as an example be used as high intensity sweeteners. Other usable high intensity sweeteners described herein may be used in combination with or instead of acesulfame potassium and/or sucralose.

Potassium sorbate is used as a preservative. Other preservatives as described herein may also be used in combination with or instead of potassium sorbate.

Silicon dioxide is used as a glidant. Other possible glidants include e.g. magnesium stearate, starch and talc.

### Example 5M

The pouch compositions are prepared from the ingredients in table 21 using preparation method described in example 3.

The pouch compositions are filled into pouches as described in example 4 (pouch material of examples 1A was used, but 1B could also have been applied).

TABLE 21
Pouch compositions.

| Pouches | P120 | P121 | P122 | P123 | P124 | P125 | P126 | P127 |
|---|---|---|---|---|---|---|---|---|
| Amount of | 9.6 mg | 9.6 mg | 9.6 mg | 9.6 mg | 9.6 mg | 9.6 mg | 9.6 mg | 9.6 mg |
| nicotine | | | | | | | | |
| Water content | 30 | 30 | 30 | 30 | 30 | 30 | 27 | 27 |
| [wt %] | | | | | | | | |
| Inorganic | 2.0 | 3.0 | 4.0 | 2.0 | 3.0 | 4.0 | 7.5 | 7.5 |
| divalent cations | | | | | | | | |
| [eq]* | | | | | | | | |
| Raw material | Content in weight percent | | | | | | | |
| Premix II | 14.6 | 14.6 | 14.6 | — | — | — | 14.6 | — |
| Premix VI | — | — | | 6.4 | 6.4 | 6.4 | — | 6.4 |
| CaCl₂** | 2.6 | 3.9 | 5.2 | 2.6 | 3.9 | 5.2 | 10.0 | 10.0 |
| Xylitol | 8.8 | 7.5 | 6.2 | 14.0 | 12.7 | 11.4 | 7.4 | 12.6 |
| Purified water | 25 | 25 | 25 | 28 | 28 | 28 | 22 | 25 |
| Wheat fiber | 30 | 30 | 30 | 30 | 30 | 30 | 27 | 27 |
| Sodium alginate | 2.0 | 2.0 | 2.0 | 2.0 | 2.0 | 2.0 | 2.0 | 2.0 |
| Sodium | 5.0 | 5.0 | 5.0 | 5.0 | 5.0 | 5.0 | 5.0 | 5.0 |
| carbonate | | | | | | | | |
| Flavor | 8.9 | 8.9 | 8.9 | 8.9 | 8.9 | 8.9 | 8.9 | 8.9 |
| High intensity | 1.0 | 1.0 | 1.0 | 1.0 | 1.0 | 1.0 | 1.0 | 1.0 |
| sweetener | | | | | | | | |
| Potassium | 0.1 | 0.1 | 0.1 | 0.1 | 0.1 | 0.1 | 0.1 | 0.1 |
| sorbate | | | | | | | | |
| Silicon dioxide | 2.0 | 2.0 | 2.0 | 2.0 | 2.0 | 2.0 | 2.0 | 2.0 |
| Total | 100 | 100 | 100 | 100 | 100 | 100 | 100 | 100 |

*The inorganic divalent cations are presented as equivalents relative to nicotine in nicotine ion-exchange combination.
**Divalent cations may be provided as a hydrated salt, such as dihydrate, tetrahydrate, hexahydrate etc. The weight % in the table are based on the non-hydrated salt.

Pouch content: 500 mg total, i.e. nicotine conc 19.2 mg/g.

Wheat fiber, trade name “Vitacel 600 WF plus”. Other fibers may be used as well, such as water-insoluble plant fibers, such as oat fibers, pea fibers, rice fiber, maize fibers, oat fibers, tomato fibers, barley fibers, rye fibers, sugar beet fibers, buckwheat fibers, potato fibers, cellulose fibers, apple fibers, cocoa fibers, powdered cellulose, bran fibers, bamboo fibers, and cellulose fiber.

Sodium alginate, glycerol and hydroxypropyl cellulose (HPC) may be used as humectants. Other humectants as described herein may also be used in combination with sodium alginate, glycerol or HPC or as an alternative.

Sodium carbonate is used as an alkaline buffering agent. Other buffering agents as described herein may also be used in combination with sodium carbonate or an alternative.

Flavor example, a mixture of e.g. menthol and peppermint may be used. Of course, other flavors as described herein may be use as well, in combination with menthol and/or peppermint or replacing these. The flavor may be liquid or flavored or a combination, i.e. a liquid flavor and a powdered flavor is added.

Acesulfame potassium and/or sucralose may as an example be used as high intensity sweeteners. Other usable high intensity sweeteners described herein may be used in combination with or instead of acesulfame potassium and/or sucralose.

Potassium sorbate is used as a preservative. Other preservatives as described herein may also be used in combination with or instead of potassium sorbate.

Silicon dioxide is used as a glidant. Other possible glidants include e.g. magnesium stearate, starch and talc.

### Example 6A: Release Experiment and Varying Salts

The release experiment was performed by adding an amount of NPR (16%) and varying equivalent of CaCl₂ to 900 mL of water corresponding to a nicotine concentration of 28 mg/L. The equivalents of CaCl₂ are relative to nicotine. The temperature of the water was 25 degrees Celsius throughout the experiment and stirring of 100 rpm was applied throughout the experiment. pH was measured at experiment start and end. The pH was in all experiments below 7.0 at both the start and end of the experiment.

A relative low nicotine concentration is used in order to reduce the impact of equilibrium on both the release rate and effective release of nicotine from the ion-exchange resin.

Samples were taken out at varying timepoints and analyzed for nicotine content using standard HPLC. The results are presented as percentage of nicotine released.

TABLE 22
Release of nicotine over time in the presence of
varying salts and varying equivalents of cations.

| | Salt | | | | |
|---|---|---|---|---|---|
| | No | 1 eq | 10 eq | 1 eq | 10 eq |
| | Salt | NaCl | NaCl | CaCl₂ | CaCl₂ |
| Minutes | Released nicotine (%) | | | | |
| 1 | 12.4 | — | — | 46.3 | — |
| 2 | 15.9 | 24.4 | 43.8 | — | 80.3 |
| 3 | — | — | — | 58.4 | — |
| 4 | 18.1 | — | — | — | — |
| 8 | 20.2 | — | — | 69.2 | — |
| 11 | 20.9 | — | — | 72.6 | — |
| 13 | — | 28.1 | 51.9 | | 89.9 |
| 14 | 21.8 | — | — | 75.0 | — |
| 17 | 22.4 | — | — | 76.5 | — |
| 20 | 23.1 | — | — | 78.2 | — |
| 23 | — | 29.9 | 52.9 | | — |
| 25 | 24.0 | — | — | | — |
| 30 | 24.8 | — | — | | — |
| 33 | — | 30.1 | 54.5 | | 90.4 |
| 35 | 25.7 | — | — | | — |
| 40 | 26.5 | — | — | | — |
| 45 | 27.2 | — | — | 81.1 | — |
| 60 | 28.8 | — | — | 82.0 | — |

Evaluation: the result shows that the presence of CaCl₂ significantly increases the release of nicotine from NPR. Increasing the amount of CaCl₂ result in an increased release of nicotine. The presence of CaCl₂ increases both the initial release rate and seems to also increase the effective release of nicotine.

Furthermore, the results show that NaCl has a much lower effect on the release of nicotine, thus high amount of NaCl are needed in order to achieve comparable release of nicotine in the presence of for example 1 eq. of CaCl₂.

### Example 6B: Release Experiment Using NPR and Varying Equivalents of CaCl₂

The release experiment was performed by adding NPR (16%) and varying equivalent of CaCl₂ to a volume of water corresponding to a nicotine concentration of 28 mg/L. The equivalents of CaCl₂ are relative to nicotine. The temperature of the water was 25 degrees Celsius throughout the experiment and stirring of 100 rpm was applied throughout the experiment. pH was measured at experiment start and end. The pH was in all experiments below 7.0 at both the start and end of the experiment.

A relative low nicotine concentration is used in order to reduce the impact of equilibrium on both the release rate and effective release of nicotine from the ion-exchange resin.

Samples were taken out at varying timepoints and analyzed for nicotine content using standard HPLC. The result is presented as percentage of nicotine released.

TABLE 23
Shows the percentage of nicotine released from NPR at different
timepoints in the presence of varying equivalent of CaCl₂.

| | CaCl₂ | | | | | | |
|---|---|---|---|---|---|---|---|
| | 0 eq | 0.1 eq | 0.25 eq | 0.5 eq | 0.75 eq | 1 eq | 4 eq |
| Minutes | Released nicotine (%) | | | | | | |
| 1 | 12.4 | 17.7 | 25.0 | 30.5 | 38.5 | 46.3 | 59.1 |
| 2 | 15.9 | 22.0 | — | 39.7 | — | — | — |
| 3 | — | — | 33.9 | — | 51.7 | 58.4 | 71.8 |
| 4 | 18.1 | 24.3 | — | 46.1 | — | — | — |
| 5 | — | — | 38.6 | — | 59.3 | — | 76.8 |
| 7 | — | — | 42.4 | — | 64.0 | — | — |
| 8 | 20.2 | 26.6 | — | 53.3 | — | 69.2 | 79.9 |
| 9 | — | — | 43.3 | — | 66.7 | — | — |
| 11 | 20.9 | 27.8 | 44.1 | 56.4 | 68.9 | 72.6 | 82.4 |
| 13 | — | — | 46.0 | — | 71.0 | — | — |
| 14 | 21.8 | 28.7 | — | 58.9 | — | 75.0 | 83.9 |
| 15 | — | — | 45.9 | — | 73.0 | — | — |
| 17 | 22.4 | 29.3 | — | 61.0 | 74.4 | 76.5 | 84.7 |
| 18 | — | — | 47.2 | — | — | — | — |
| 20 | 23.1 | 30.3 | 47.5 | 62.4 | 76.3 | 78.2 | 85.0 |
| 25 | 24.0 | 31.1 | — | 64.4 | — | — | — |
| 30 | 24.8 | 31.8 | 49.3 | 65.8 | — | — | — |
| 35 | 25.7 | 32.6 | — | 66.8 | — | — | — |
| 40 | 26.5 | 33.2 | — | 67.8 | — | — | — |
| 45 | 27.2 | 33.8 | 50.6 | 69.2 | 80.2 | 81.1 | 87.3 |
| 60 | 28.8 | 35.0 | 51.7 | 69.2 | 81.1 | 82.0 | 88.1 |

Evaluation: the result shows that the presence of CaCl₂ significantly increases the release of nicotine from NPR. Increasing the amount of CaCl₂ result in an increased release of nicotine. The presence of CaCl₂ increases both the initial release rate and seems to also increase the effective release of nicotine.

### Example 6C: Release Experiment Using NPR and Varying Equivalents of MgCl₂

The release experiment was performed by adding NPR (16%) and varying equivalents of MgCl₂ to a volume of water corresponding to a nicotine concentration of 28 mg/L. The equivalents of MgCl₂ are relative to nicotine. The temperature of the water was 25 degrees Celsius throughout the experiment and stirring of 100 rpm was applied throughout the experiment. pH was measured at experiment start and end. The pH was in all experiments below 7.0 at both the start and end of the experiment.

A relative low nicotine concentration is used in order to reduce the impact of equilibrium on both the release rate and effective release of nicotine from the ion-exchange resin.

Samples were taken out at varying timepoints and analyzed for nicotine content using standard HPLC. The result is presented as percentage of nicotine released.

TABLE 24
Shows the percentage of nicotine released from NPR at different
timepoints in the presence of varying equivalents of MgCl₂.

| | MgCl₂ | | | | | | | |
|---|---|---|---|---|---|---|---|---|
| Min- | 0 eq | 0.1 eq | 0.25 eq | 0.5 eq | 0.75 eq | 1 eq | 2 eq | 4 eq |
| utes | Released nicotine (%) | | | | | | | |
| 1 | 12.4 | 16.8 | 23.2 | 33.7 | 40.6 | 42.3 | 53.7 | 63.0 |
| 3 | — | 22.9 | 32.2 | 44.1 | 52.2 | 55.3 | 66.5 | 73.6 |
| 5 | — | 25.8 | 37.0 | 49.9 | 58.1 | 62.4 | 72.2 | 79.4 |
| 7 | — | 27.6 | 39.9 | 54.0 | 62.4 | 66.7 | 74.8 | 81.3 |
| 9 | — | 28.4 | 41.6 | 56.7 | 64.8 | 69.3 | 76.5 | 83.2 |
| 11 | 20.9 | 29.1 | 43.0 | 58.6 | 67.5 | 71.7 | 78.2 | 83.9 |
| 13 | — | 29.9 | 44.5 | 60.2 | 70.1 | 73.0 | 79.7 | 85.1 |
| 15 | — | 30.5 | 44.8 | 61.6 | 71.2 | 74.2 | 80.4 | 87.0 |
| 20 | 23.1 | 31.5 | 47.2 | 64.5 | 72.8 | 76.5 | 82.1 | 87.5 |
| 25 | 24.0 | 32.5 | 47.7 | 65.7 | 75.8 | 77.7 | 83.8 | 87.9 |
| 30 | 24.8 | 33.2 | 48.8 | 68.1 | 78.2 | — | — | 88.1 |

Evaluation: the result shows that the presence of MgCl₂ significantly increases the release of nicotine from NPR. Increasing the amount of MgCl₂ result in an increased release of nicotine. The presence of MgCl₂ increases both the initial release rate and seems to also increase the effective release of nicotine. The results are comparable to the result presented in example 6B.

### Example 6D: Release Experiment Using 1 Equivalent of CaCl₂ and Nicotine Premix Having Varying Content of Nicotine

The release experiment was performed by adding nicotine premix having varying content of nicotine and 1 equivalent of CaCl₂ to a volume of water, whereby a corresponding nicotine concentration of 28 mg/L is obtained. The equivalent of CaCl₂ is relative to nicotine. The temperature of the water was 25 degrees Celsius throughout the experiment and stirring of 150 rpm was applied throughout the experiment. pH was measured at experiment start and end. The pH was in all experiments below 7.0 at both the start and end of the experiment.

A relative low nicotine concentration is used in order to reduce the impact of equilibrium on both the release rate and effective release of nicotine from the ion-exchange resin.

Samples were taken out at varying timepoints and analyzed for nicotine content using standard HPLC. The result is presented as percentage of nicotine released.

TABLE 25
Shows the percentage of nicotine released from nicotine premix at
different timepoints in the presence of 1 equivalent of MgCl₂.
Ingredients

| | Premix | | | | | | | |
|---|---|---|---|---|---|---|---|---|
| | II | II | VI | VI | VII | VII | VIII | VIII |
| | CaCl₂ | | | | | | | |
| | — | 1 eq. | — | 1 eq. | — | 1 eq. | — | 1 eq. |
| Min. | Released nicotine (%) | | | | | | | |
| 1 | 2.1 | 9.1 | 37.2 | 56.3 | 43.8 | 55.7 | 58.3 | 69.6 |
| 2 | 3.0 | 14.7 | 44.9 | 66.9 | 53.4 | 66.0 | 66.2 | 78.7 |
| 3 | 4.0 | 19.9 | 48.9 | 71.4 | 57.4 | 73.4 | 70.1 | 82.6 |
| 4 | 4.9 | 24.7 | 51.8 | 76.8 | 60.1 | 77.8 | 72.0 | 85.9 |
| 5 | 5.7 | 29.8 | 53.7 | 79.5. | 62.2 | 81.4 | 73.0 | 88.4 |
| 6 | 6.5 | 33.7 | 54.4 | 81.6 | 63.0 | 84.1 | 74.7 | 90.3 |
| 7 | 7.1 | 38.6 | 55.4 | 83.1 | 64.4 | 86.2 | 75.0 | 92.3 |
| 8 | 8.0 | 42.0 | 56.1 | 84.8 | 65.2 | 88.9 | 75.4 | 92.9 |
| 9 | 8.4 | 46.4 | 56.9 | 86.3 | 65.5 | 90.5 | 75.7 | 94.5 |
| 10 | 9.0 | 49.9 | 57.4 | 87.3 | 66.1 | 91.1 | 76.0 | 94.7 |
| 11 | — | 53.1 | 58.0 | 88.0 | 66.5 | 92.6 | 76.5 | 95.7 |
| 12 | — | 55.6 | 58.4 | 89.2 | 67.3 | 93.4 | — | 96.3 |
| 13 | — | 57.7 | 58.5 | 89.9 | 66.9 | 93.8 | 77.0 | 96.9 |
| 14 | — | 60.5 | 58.9 | 90.9 | 67.3 | 95.0 | — | 97.3 |
| 15 | 11.8 | 62.0 | 59.6 | 91.6 | 68.2 | 95.8 | 77.3 | 97.3 |

Evaluation: the result shows that the presence of CaCl₂ significantly increases the release of nicotine from premixes. The presence of CaCl₂ increases both the initial release rate and seems to also increase the effective release of nicotine. Furthermore, the results demonstrate that increasing the nicotine content of the premixes also increases the nicotine release.

### Example 6E: Release Experiment Using 1 Equivalent of AlCl₃ or 1 Equivalent of MgO

The release experiment was performed by adding NPR (16%) and 1 equivalent of AlCl₃ to a volume of water corresponding to a nicotine concentration of 28 mg/L. The equivalents are relative to nicotine. The temperature of the water was 25 degrees Celsius throughout the experiment and stirring of 150 rpm was applied throughout the experiment. pH was measured at experiment start and end. The pH was in all experiments below 7.0 at both the start and end of the experiment.

A relative low nicotine concentration is used in order to reduce the impact of equilibrium on both the release rate and effective release of nicotine from the ion-exchange resin.

Samples were taken out at varying timepoints and analyzed for nicotine content using standard HPLC. The result is presented as percentage of nicotine released.

TABLE 24
Shows the percentage of nicotine released from NPR at different
timepoints in the presence of 1 equivalent of AlCl₃.

Salt

| | |
|---|---|
| No Salt | 1 eq AlCl₃ |

| | |
|---|---|
| Minutes | Released nicotine (%) |

| | | |
|---|---|---|
| 1 | 11.1 | 39.9 |
| 3 | 14.5 | 49.4 |
| 5 | 16.1 | 55.4 |
| 8 | 18.2 | 60.9 |
| 11 | 19.7 | 64.4 |
| 15 | 20.0 | 68.4 |
| 20 | 21.1 | 71.4 |
| 25 | 21.5 | 74.0 |
| 30 | 22.4 | 75.4 |

Evaluation: the results demonstrate that the presence of 1 equivalent of AlCl₃ significantly increases the release of nicotine from NPR. The presence of AlCl₃ increases both the initial release rate and seems to also increase the effective release of nicotine.

### Example 7A: Pouch Release Experiments (In Vitro)

The release properties of the pouches were tested in an in vitro experiment.

Reaction tubes having a diameter approx. 2 cm and containing 10 mL of 0.02 M potassium dihydrogen phosphate-buffer (pH adjusted to 7.4) were warmed to 37 degrees Celsius. One reaction tuber per timepoint was used.

A pouch was submerged in the buffer of the first reaction tube using tweezers. After a specified time period, the pouch was captured with the tweezer and gently swirled in the buffer before being removed from the first reaction tube and added to the next reaction tube, representing the next time point. The procedure was repeated until the desired number of time points had been tested.

The whole release experiment was performed at 37 degrees Celsius. No stirring or shaken was applied during the release experiment.

The amount of release nicotine was determined by analyzing the buffer samples at the different timepoints using standard HPLC.

### Example 8A: Release Experiment on Pouches

The release experiment was performed as described in example 7A.

TABLE 27
Shows the percentage of nicotine released from nicotine pouches at different
timepoints in the presence of varying equivalents of CaCl₂.

| | Pouch | | | | | | | | | |
|---|---|---|---|---|---|---|---|---|---|---|
| | C4 | P40 | P42 | C5 | P43 | P45 | C10 | P110 | C11 | P113 |
| | Premix | | | | | | | | | |
| | II | II | II | VI | VI | VI | II | II | VI | VI |
| | CaCl₂ | | | | | | | | | |
| | — | 0.75 eq | 1.5 eq | — | 0.75 eq | 1.5 eq | — | 0.75 eq | — | 0.75 eq |
| | NaCl | | | | | | | | | |
| | 2.9 eq | — | — | 2.9 eq | — | — | — | — | — | — |
| Min. | Released nicotine (%) | | | | | | | | | |
| 2 | 13.8 | 12.9 | 32.4 | 20.8 | 24.0 | 39.3 | 16.2 | 29.5 | 26.9 | 38.7 |
| 5 | 25.7 | 26.0 | 49.6 | 39.8 | 42.9 | 62.5 | 28.6 | 50.9 | 47.4 | 58.2 |
| 10 | 37.5 | 40.3 | 66.0 | 59.0 | 61.7 | 78.8 | 42.0 | 64.8 | 66.0 | 74.8 |
| 30 | 60.4 | 62.3 | 79.9 | 79.4 | 82.6 | 90.3 | 59.2 | 78.7 | 79.5 | 92.2 |

Evaluation: comparing P110 and P113 with C10 and C11 respectively, the result shows that the presence of CaCl₂ increases the release of nicotine from pouches. The presence of CaCl₂ increases both the initial release rate and seems to also increase the effective release of nicotine. Comparing P40 and P42, demonstrate that increasing the amount of CaCl₂ in a pouch also increases the nicotine release from the pouch.

Furthermore, the results demonstrate that increasing the nicotine content of the premixes also increases the nicotine release from the pouches, comparing P40 with P43, P42 with P45 and P110 with P113.

Finally, it is noted that in order to obtain a release being comparable to the release obtained from pouches comprising only 0.75 eq CaCl₂, a much higher amount of NaCl will be needed, here at least 2.9 eq NaCl required to obtain a release being comparable to 0.75 eq CaCl₂ (see C4, C5, P40 and P43).

### Example 9A: User Evaluation

The produced pouches of the invention were evaluated and found highly suitable as delivery vehicles of nicotine in that they provide a favorable release of nicotine and at the same time are pleasant to the user, e.g. with respect to a desirable mouthfeel such as a moist and moldable texture and a desirable taste.

### Example 9B: User Evaluation

The pouch product P03 and P44 was evaluated with respect to perceived effect from nicotine and mouthfeel.

Evaluation of perceived effect from nicotine and mouthfeel is performed as described in the following.

Perceived effect from nicotine and mouthfeel was evaluated by a test panel of 4 trained assessors. Each assessor evaluates all samples twice. Average evaluations are estimated.

The pouch product P03 and P44 were evaluated to have a fast onset of action and a high perceived effect from nicotine by all four assessors. Also, all four assessors evaluated the pouch products to have a desirable mouthfeel, i.e. the pouches were found to be moist and have a desirable taste.

Similarly, the pouches, P08 and P127, were evaluated. These pouches were evaluated to have a fast onset of action and a high perceived effect from nicotine by all four assessors. However, the pouches were found to provide a less desirable mouthfeel, the pouches being perceived as somewhat dry, adhering to the oral mucosa and/or as having a poor taste or less desirable taste, i.e. too salty.

It should be noted that the invention in its broader aspects is not limited to the specific details, representative compositions, methods, and processes, and illustrative examples described in connection with the preferred embodiments and preferred methods. Modifications and equivalents will be apparent to practitioners skilled in this art and are encompassed within the spirit and scope of the appended claims.

1. A pouch composition comprising
a nicotine-ion exchange resin combination,
water in an amount of at least 15% by weight of the pouch composition, and
inorganic divalent cations.

2. The pouch composition according to claim 1, wherein the inorganic divalent cations are in a molar ratio of at least 0.1 relative to amount of nicotine in the nicotine-ion exchange resin combination.

3. The pouch composition according to claim 1, wherein the composition comprises inorganic divalent cations in a molar ratio of at most 5 relative to amount of nicotine in the nicotine-ion exchange resin combination.

4. The pouch composition according to claim 1, wherein the inorganic divalent cations are selected from the group consisting of divalent cations of calcium, magnesium, iron, zinc, and any combination thereof.

5. The pouch composition according to claim 1, wherein the inorganic divalent cations are selected from the group consisting of divalent cations of calcium and magnesium.

6. The pouch composition according to claim 1, wherein the inorganic divalent cations are provided as a salt comprising anions selected from the group consisting of carboxylates, organic sulfonate, organic sulfate, organic phosphate, chloride, bromide, nitrate, sulfate, hydrogen phosphate, oxide, and any combination thereof.

7. (canceled)

8. The pouch composition according to claim 1, wherein the inorganic divalent cations are provided as a salt in an amount of between 0.1 and 15.0% by weight of the composition.

9. The pouch composition according to claim 1, wherein the inorganic divalent cations are provided as an inorganic salt comprising inorganic anions selected from the group consisting of chloride, bromide, nitrate, sulfate, hydrogen carbonate, hydrogen phosphate, oxide, hydroxide, and any combination thereof.

10-13. (canceled)

14. The pouch composition according to claim 1, wherein the divalent cations are provided as a water-soluble salt having a water-solubility of at least 5 gram per 100 mL of water measured at 25 degrees Celsius, atmospheric pressure and pH 7.0.

15. The pouch composition according to claim 1, wherein the pouch composition comprises nicotine in an amount of at least 0.1% by weight.

16. The pouch composition according to claim 1, wherein the pouch composition comprises the nicotine-ion exchange combination in an amount of 0.1 to 20% by weight of the pouch composition.

17-19. (canceled)

20. The pouch composition according to claim 1, wherein the ion exchange resin comprises polacrilex resin.

21. (canceled)

22. The pouch composition according to claim 1, wherein the nicotine-ion exchange resin combination comprises nicotine complexed with ion exchange resin.

23. (canceled)

24. The pouch composition according to claim 1, wherein the nicotine-ion exchange resin combination comprises free-base nicotine mixed with ion exchange resin.

25. (canceled)

26. The pouch composition according to claim 1, wherein the pouch composition comprises at least one sugar alcohol.

27. The pouch composition according to claim 26, wherein the at least one sugar alcohol is selected from xylitol, maltitol, mannitol, erythritol, isomalt, sorbitol, lactitol, and mixtures thereof.

28-30. (canceled)

31. The pouch composition according to claim 1, wherein the pouch composition comprises at least one water-insoluble fiber.

32-33. (canceled)

34. The pouch composition according to claim 31, wherein the water-insoluble fiber is selected from wheat fibers, pea fibers, rice fiber, maize fibers, oat fibers, tomato fibers, barley fibers, rye fibers, sugar beet fibers, buckwheat fibers, potato fibers, cellulose fibers, apple fibers, cocoa fibers, cellulose fibers, bran fibers, bamboo fibers, powdered cellulose, and combinations thereof.

35-36. (canceled)

37. The pouch composition according to claim 1, wherein the pouch composition comprises a pH regulating agent.

38-50. (canceled)

51. An oral pouched nicotine product comprising a saliva-permeable pouch and the pouch composition of claim 1 enclosed in said pouch.

52-58. (canceled)
